(19) United States
(12) Patent Application Publication
(10) Pub. No.: US 2010/0152619 A1
(43) Pub. Date: Jun. 17, 2010

(54) SYSTEM, METHOD, AND COMPUTER-PROGRAM PRODUCT FOR MEASURING PRESSURE POINTS

(75) Inventors: Alex J. Kalpaxis, Glendale, NY (US); David Schieffelin, Woodbury, CT (US); Stacey S. Schieffelin, Woodbury, CT (US); Tracey L. Stetler, Cos Cob, CT (US)

Correspondence Address: VENABLE LLP, P.O. BOX 34385, WASHINGTON, DC, 20043-9998, US

(73) Assignee: 24/8 LLC, New York, NY (US)

(21) Appl. No.: 12/336,088

(22) Filed: Dec. 16, 2008

(51) Int. Cl. A61B 5/103; A63F 9/24; G09B 19/00

(52) U.S. Cl. 600/592; 463/36; 434/258

(57) ABSTRACT

Force sensing methods, systems, and computer-program products may be used to sense pressure at a plurality of points of a user's foot, including its bones, joints, muscles, tendons, and ligaments. Such systems, methods, and computer-program products sense pressure along the bottom of a user's foot during sports training and monitoring applications, electronic games, and diagnostic systems. In particular, the system generally comprises a transducer having a plurality of points of interest, an insole node for collecting and transmitting data sensed at the plurality of points of interest, first means for coupling that data across a network, by way of second means for coupling same to a collector node, and then to a computer.

### CROSS-REFERENCE TO RELATED APPLICATIONS

This application claims the benefit of the following related applications: application Ser. No. 12/155,558, filed on Jun. 5, 2008, which, in turn, claims the benefit of application Ser. No. 60/924,931, filed on Jun. 5, 2007, and application Ser. No. 60/996,608, filed on Nov. 27, 2007; and PCT/US08/85065, filed on Nov. 28, 2008, each of which is incorporated herein by reference in its entirety.

### BACKGROUND OF THE INVENTION

1. Field of the Invention

The present invention in its disclosed embodiments is related generally to pressure sensing systems, methods, and computer-program products, and more particularly to such systems, methods, and computer-program products for sensing pressure along the bottom of a user's foot during sports training and monitoring applications, electronic games, and diagnostic systems as will become more apparent hereinafter. However, it should be readily appreciated to those of ordinary skill in the art that the following embodiments may also be applicable to the other pressure sensing applications.

2. Statement of the Prior Art

Athletes utilize various metrics to measure their performance and chart their workouts. The metrics are recorded and analyzed both during and after workouts. For example, interval type workouts typically involve multiple sets of intense activity, semi-intense activity, and rest. The intense activity may be characterized by a range of metrics which correlate to the desired intensity for a particular athlete. Likewise, the rest or semi-intense activity periods may be characterized by a range or metrics which correlate to the desired restful state for a particular athlete.

Accordingly, it would be desirable to provide systems, methods, and computer-program products for sensing pressure along the bottom of a user's foot, to record and analyze such metrics during sports training and monitoring applications, electronic games, and diagnostic systems.

The human foot combines mechanical complexity and structural strength. The ankle serves as foundation, shock absorber, and propulsion engine. The foot can sustain enormous pressure (i.e., in the range of about several tons over the course of a one-mile run) and provides flexibility and resiliency.

The foot and ankle contain 26 bones (i.e., nearly one-quarter of the bones in the human body are in the feet); 33 joints; more than 100 muscles, tendons (i.e., fibrous tissues that connect muscles to bones), and ligaments (i.e., fibrous tissues that connect bones to other bones); and a network of blood vessels, nerves, skin, and soft tissue.

These components work together to provide the body with support, balance, and mobility. A structural flaw or malfunction in anyone part can result in the development of problems elsewhere in the body. Abnormalities in other parts of the body can lead to problems in the feet. Embodiments of the present invention help sense the pressure exerted at a plurality of points of the user's feet to help alleviate such problems.

Structurally, the foot has three main parts: the forefoot, the midfoot, and the hindfoot. The forefoot as shown in FIGS. 2A and 2B is composed of the five toes (called phalanges) and their connecting long bones (metatarsals). Each toe (phalanx) is made up of several small bones. The big toe (also known as the hallux) has two phalanx bones-distal and proximal. It has one joint, called the interphalangeal joint. The big toe articulates with the head of the first metatarsal and is called the first metatarsophalangeal joint (MTPJ for short). Underneath the first metatarsal head are two tiny, round bones called sesamoids. The other four toes each have three bones and two joints. The phalanges are connected to the metatarsals by five metatarsal phalangeal joints at the ball of the foot. The forefoot bears half the body's weight and balances pressure on the ball of the foot.

The midfoot has five irregularly shaped tarsal bones, forms the foot's arch, and serves as a shock absorber. The bones of the midfoot are connected to the forefoot and the hindfoot by muscles and the plantar fascia (arch ligament).

The hindfoot is composed of three joints and links the midfoot to the ankle (talus). The top of the talus is connected to the two long bones of the lower leg (tibia and fibula), forming a hinge that allows the foot to move up and down. The heel bone (calcaneus) is the largest bone in the foot. It joins the talus to form the subtalar joint. The bottom of the heel bone is cushioned by a layer of fat.

A network of muscles, tendons, and ligaments supports the bones and joints in the foot. There are 20 muscles in the foot that give the foot its shape by holding the bones in position and expand and contract to impart movement. The main muscles of the foot are: the anterior tibial, which enables the foot to move upward; the posterior tibial, which supports the arch; the peroneal tibial, which controls movement on the outside of the ankle; the extensors, which help the ankle raise the toes to initiate the act of stepping forward; and the flexors, which help stabilize the toes against the ground. Smaller muscles enable the toes to lift and curl.

There are elastic tissues (tendons) in the foot that connect the muscles to the bones and joints. The largest and strongest tendon of the foot is the Achilles tendon, which extends from the calf muscle to the heel. Its strength and joint function facilitate running, jumping, walking up stairs, and raising the body onto the toes. Ligaments hold the tendons in place and stabilize the joints. The longest of these, the plantar fascia, forms the arch on the sole of the foot from the heel to the toes. By stretching and contracting, it allows the arch to curve or flatten, providing balance and giving the foot strength to initiate the act of walking. Medial ligaments on the inside and lateral ligaments on outside of the foot provide stability and enable the foot to move up and down. Skin, blood vessels, and nerves give the foot its shape and durability, provide cell regeneration and essential muscular nourishment, and control its varied movements.

Pressure sensing methods, systems, and computer-program products in particular may be used to sense pressure at a plurality of points of a user's foot, including its bones, joints, muscles, tendons, and ligaments.

### SUMMARY OF THE INVENTION

These and other objects, advantages, and novel features according to embodiments of the present invention are accomplished by a sensing system generally comprising a transducer having a plurality of points of interest, first means for collecting and transmitting data sensed at the plurality of points of interest, first means for coupling that data across a network by way of second means coupling same to a collector node and then to a computer for analysis.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

“Software” may refer to prescribed rules to operate a computer. Examples of software may include: code segments in one or more computer-readable languages; graphical and or/textual instructions; applets; pre-compiled code; interpreted code; compiled code; and computer programs.

A “computer-readable medium” may refer to any storage device used for storing data accessible by a computer. Examples of a computer-readable medium may include: a magnetic hard disk; a floppy disk; an optical disk, such as a CD-ROM and a DVD; a magnetic tape; a flash memory; a memory chip; and/or other types of media that can store machine-readable instructions thereon.

A “computer system” may refer to a system having one or more computers, where each computer may include a computer-readable medium embodying software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those made through telephone or other communication links. A network may further include hard-wired connections (e.g., coaxial cable, twisted pair, optical fiber, waveguides, etc.) and/or wireless connections (e.g., radio frequency waveforms, free-space optical waveforms, acoustic waveforms, etc.). Examples of a network may include: an internet, such as the Internet; an intranet; a local area network (LAN); a wide area network (WAN); and a combination of networks, such as an internet and an intranet.

Exemplary networks may operate with any of a number of protocols, such as Internet protocol (IP), asynchronous transfer mode (ATM), and/or synchronous optical network (SONET), user datagram protocol (UDP), IEEE 802.x, etc.

Embodiments of the present invention may include apparatuses for performing the operations disclosed herein. An apparatus may be specially constructed for the desired purposes, or it may comprise a general-purpose device selectively activated or reconfigured by a program stored in the device.

Embodiments of the invention may also be implemented in one or a combination of hardware, firmware, and software. They may be implemented as instructions stored on a machine-readable medium, which may be read and executed by a computing platform to perform the operations described herein.

In the following description and claims, the terms “computer program medium” and “computer readable medium” may be used to generally refer to media such as, but not limited to, removable storage drives, a hard disk installed in hard disk drive, and the like. These computer program products may provide software to a computer system. Embodiments of the invention may be directed to such computer program products.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

In the following description and claims, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In a similar manner, the term “processor” may refer to any device or portion of a device that processes electronic data from registers and/or memory to transform that electronic data into other electronic data that may be stored in registers and/or memory. A “computing platform” may comprise one or more processors.

### BRIEF DESCRIPTION OF THE DRAWINGS

The foregoing and other features of the present invention will become more apparent from the following description of exemplary embodiments, as illustrated in the accompanying drawings wherein like reference numbers generally indicate identical, functionally similar, and/or structurally similar elements. Usually, the left most digit in the corresponding reference number will indicate the drawing in which an element first appears.

FIG. 1 illustrates a sensing system according to a first embodiment of the present invention;

FIGS. 2A and 2B illustrate parts of the human foot, some of which may be sensed by the sensing system shown in FIG. 1;

FIG. 3 illustrates a portion of the sensing system shown in FIG. 1, with an exploded view of a transducer according thereto;

FIG. 4 illustrates in schematic format electrode grid selector mapping means which may be used with the transducer shown in FIG. 3;

FIG. 5 illustrates in schematic format a controller which incorporates the electrode grid selector mapping means shown in FIG. 4;

FIG. 6 illustrates a graph showing the dependency of the resistance of the transducer as a function of pressure sensed by same;

FIG. 7 illustrates a flowchart of the transmission of data sensed by the sensing system of FIGS. 1 and 3-6;

FIG. 8 illustrates a first electrode grid layer of a transducer according to another embodiment of the present invention;

FIG. 9 illustrates a second electrode grid layer which may be used with the first electrode grid layer of a transducer according to another embodiment of the present invention;

FIGS. 10A-10F illustrate in schematic format portions of an RF module which may be used with the first and second electrode grid layers shown in FIGS. 8 and 9;

FIGS. 11 and 12 illustrate a first and a second algorithm which may be used with transducers according to FIGS. 8, 9 and 10A-10F; and

FIG. 13 illustrates the process of capturing insole data according to the embodiments described herein.

### DETAILED DESCRIPTION OF THE EMBODIMENTS

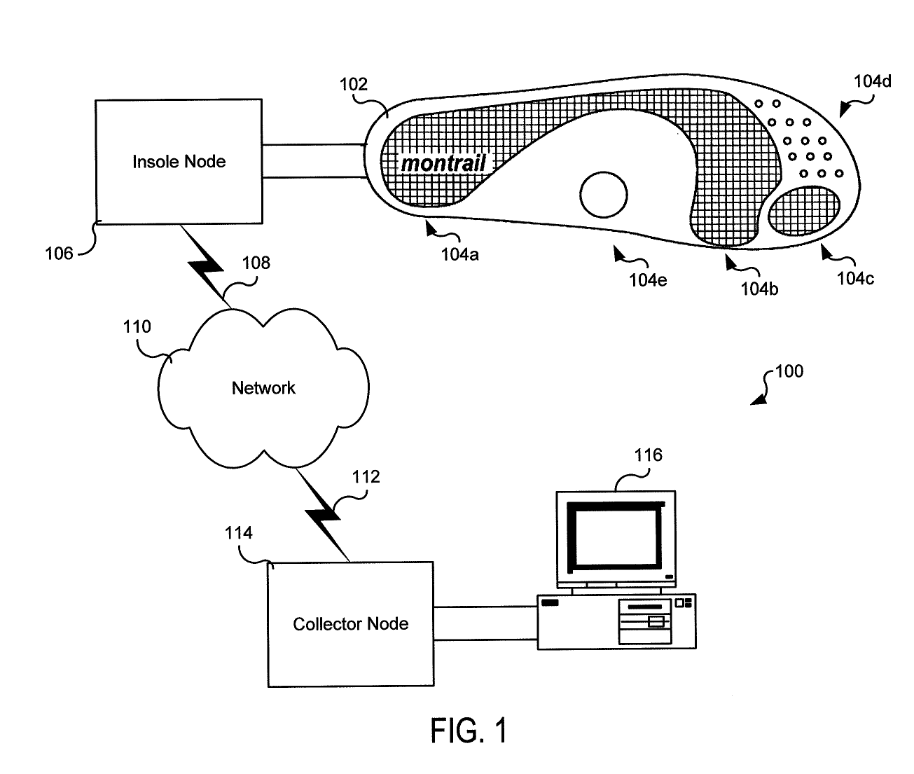

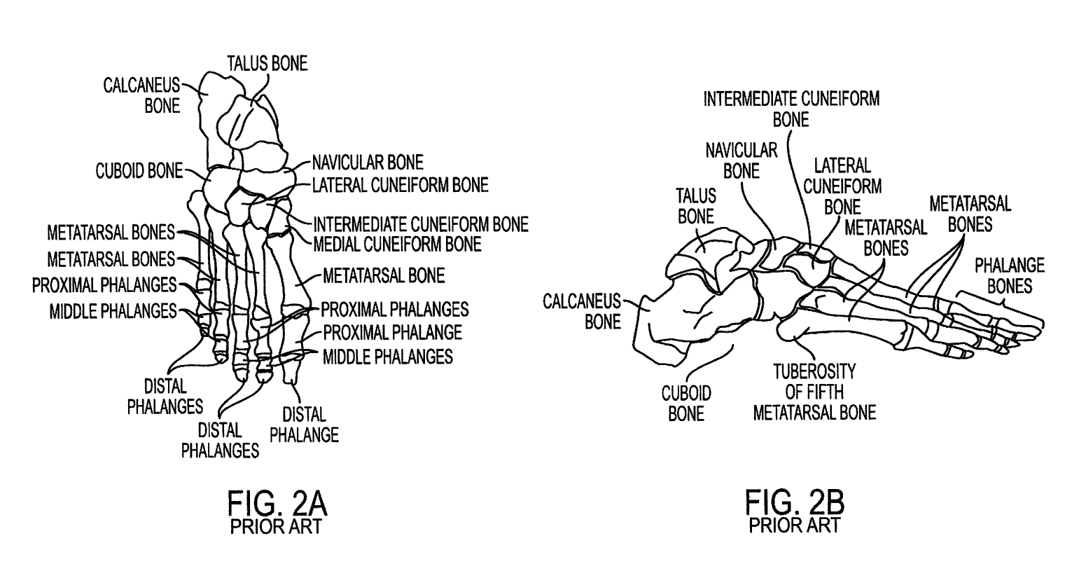

Exemplary embodiments are discussed in detail below. While specific exemplary embodiments are discussed, it should be understood that this is done for illustration purposes only. In describing and illustrating the exemplary embodiments, specific terminology is employed for the sake of clarity. However, the embodiments are not intended to be limited to the specific terminology so selected. Persons of ordinary skill in the relevant art will recognize that other components and configurations may be used without departing from the true spirit and scope of the embodiments. It is to be understood that each specific element includes all technical equivalents that operate in a similar manner to accomplish a similar purpose. Therefore, the examples and embodiments described herein are non-limiting examples. Referring now to the drawings, wherein like reference numerals and characters represent like or corresponding parts and steps throughout each of the many views, there is shown in FIG. 1 a sensing system 100 according to a first embodiment of the present invention. System 100 generally comprises a transducer 102 having a plurality of points of interest 104a, 104b, 104c, 104d, 104e, an insole node 106 for collecting and transmitting data sensed at the plurality of points of interest 104a through 104e, first means 108 for coupling that data across a network 110, by way of second means 112 for coupling same to a collector node 114, and then to a computer 116.

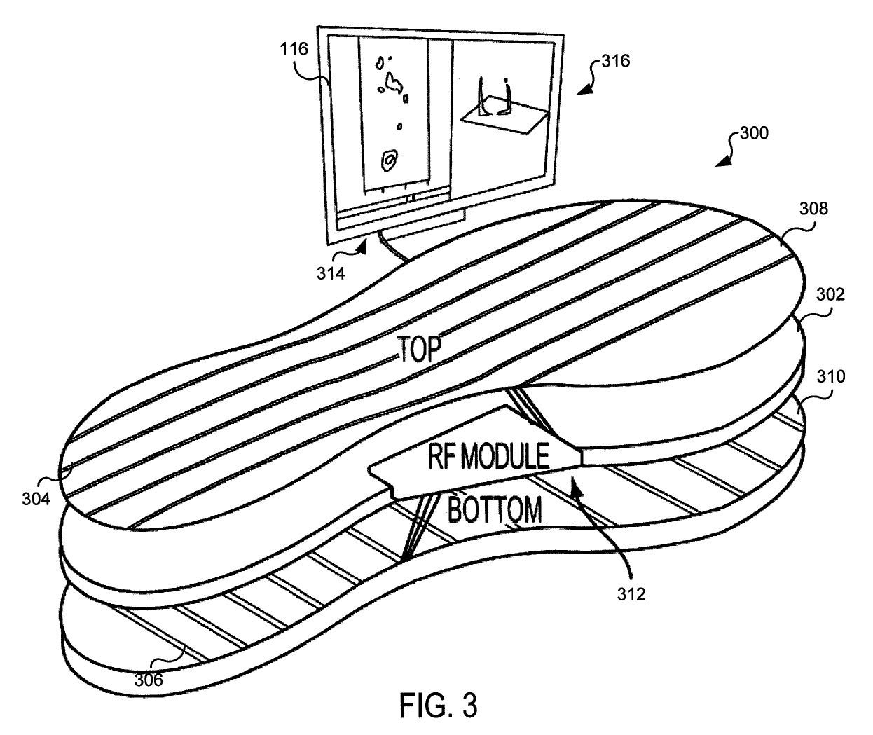

FIG. 3 illustrates a portion of the sensing system 100 shown in FIG. 1, with an exploded view of a transducer 300 according thereto. The insole of sensing system 100 comprises a foot force transducer 300 which may include a continuous capacitance pressure sensor system. Conventional foot force transducers have discrete arrays of capacitors formed by overlapping two sets of conducting strips laid in orthogonal directions on opposite sides of a center layer in a three-layer configuration.

Unlike such conventional transducers, the design of sensing system 100 allows for flexible placement of conductive elements when creating the typical three-layer configuration. The continuous capacitance pressure sensor elements of the insoles are made using a pressure sensitive variable conductive polymer 302 between conductive traces 304, 306 on sheets 308, 310 of flexible circuit made of a flexible polymer film laminated to a thin sheet of copper that is etched to produce the conductor patterns. This polyimide film is high heat resistance, has dimensional stability, good dielectric strength, with high flexibility, which allows it to survive hostile environments.

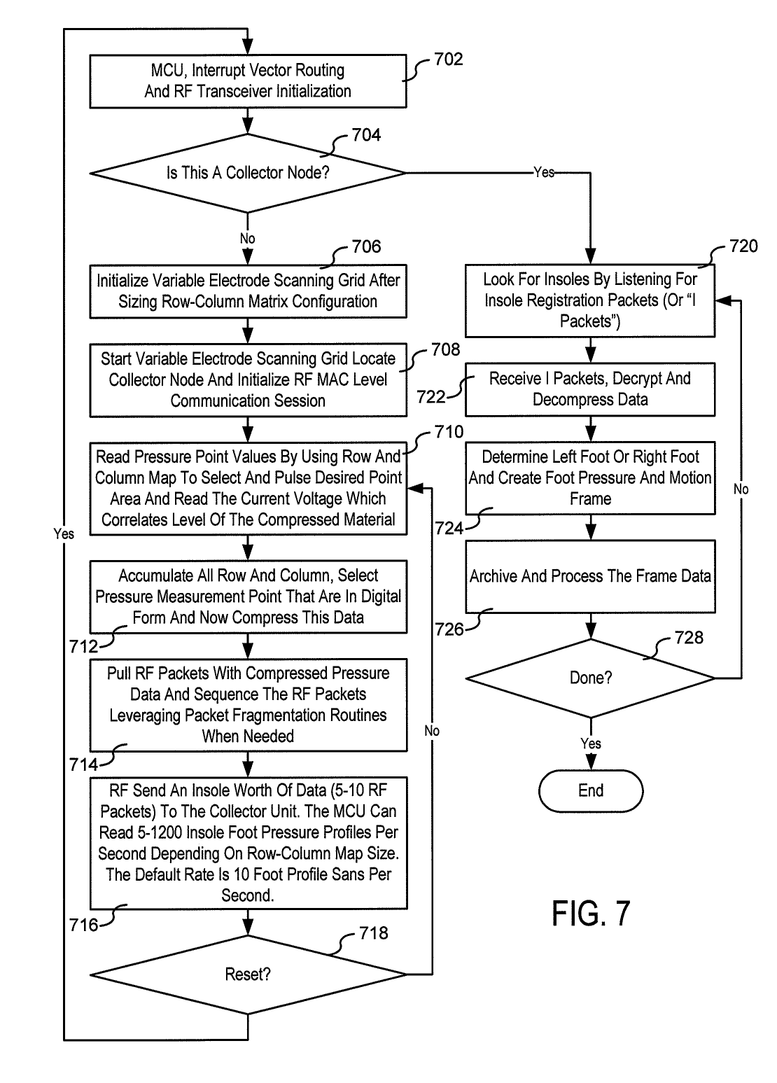

The continuous resistive/capacitive sensor layer may be an extruded electrostatic discharge (ESD) type ultra high-density conductive XPU foam. This is used to protect against very-high voltage ESD and provide a compressible form factor for physical device protection against movement shock. The material provides linear resistive and capacitive characteristics through a range of compression forces (0-30 psi) as shown in FIG. 6.

The XPU foam used in layer 302 is a semiconductor that changes its characteristic impedance as a function of applied pressure or compression. As FIG. 6 shows, the impedance characteristics of the material are non-linear at low applied pressures (e.g., less than 10 psi) or compression, and become linear as the applied pressure or compression increases. Thicknesses of the XPU material is also a determining factor of characteristic XPU material impedance. The impedance function is characterized by:

Z(p) XPU=[R(p)+jwC(p)](1−exp(−p/Rc)) for 0<=p<=Pc

Z(p) XPU=R(p)+jwC(p) for Pc<p<Pmax

Z(p) XPU≈R(P) for Po<p<30 psi Po=Pc*n*0.125

where n=number of XPU layers (i.e., thickness=0.125 inches) and P is the operating linear starting pressure. The characteristic impedance of the XPU material may be profiled algorithmically with embedded software running on a processor of sensing system 100.

It may be appreciated that variable pressure analysis point techniques may be used to dynamically map a plurality of points/regions of interest for the foot pressure measurement. For instance, and referring again to FIG. 1, a portion of the heel area 104a and the toe areas 104c, 104d may be measured for approximately 10 milliseconds, while an arch area 104e may be measured for 25 milliseconds. This would allow for pattern measurements, for instance, in the case of persons with diabetes, where the nerve damage as a result of the disease does not allow the person to become aware of the fact that certain areas of the feet are swelling. By using targeted pattern measurement, alerts to changes in plantar foot pressure variations may be provided.

It is contemplated that other materials such as piezoceramic materials which may provide capacitive, piezoelectric, and/or resistive effects may also be used.

The transducer 300 of sensing system 100 incorporates these modular lightweight, high resolution, continuous pressure sensing shoe insoles, which may be reconfigurable for varying arrangements, to wirelessly transmit through an RF module 312, detailed pressure data to a computer 116, where the data may be collated and collectively displayed. Sensing system 100 may be integrated with other systems (e.g., vision based sensing systems) to provide robust multi-modal sensing capabilities. Sensing system 100, thus, not only provides a series of applications for data analysis/visualization, data recording and playback, but also may be grouped together to form clusters of sensing systems that send real-time data to computers.

Sensing system 100 detects the changes in the electrical properties of continuous capacitance pressure sensors, caused by the mechanical deformation of its material. It may have typical recording durations of about one second at a sampling rate of 50 Hz for a transducer 300 comprising 200 elements, which results in about 10,000 pressure data points per transducer per second. With this volume of information, visual presentation and data reduction techniques may be used. The graphical representation of pressure distribution may be through wire frame diagrams 314. These pressure maps are obtained for each sampling interval or at specific instants during the foot-ground contact. A peak pressure graphical representation 316 may also be used to illustrate individual foot contact behavior with the ground. This image may be created by presenting the highest pressures under the foot, as they have occurred at any time during the ground contact.

Sensing system 100 is able to measure plantar pressure during bipedal standing, which results in about 2.6 times higher heel against forefoot pressures. The highest forefoot pressures are located under the second and third metatarsal heads. There is almost no load sharing contribution of the toes during this standing period. The peak plantar pressures indicate no substantial relationship to body weight. Sensing system 100 measures foot pressures during bipedal standing, walking, and running and shows the highest pressures under the forefoot are found under the third metatarsal head. For bipedal standing as well as walking, peak pressures beneath the third metatarsal head are substantially higher than under the other metatarsal heads. When running, during the impact phase of the ground reaction force, the momentum from the decelerating limb rapidly changes as the foot collides with the ground, resulting in a transient force transmitted up the skeleton. These forces reach magnitudes of up to three times body weight. The repetitive transmission of these forces contributes to degradation and overuse injuries. The ability of sensing system 100 to measure plantar pressure distributed over the sole of a foot during running allows for an early determination of potential degradation and overuse injury by profiling the foot's biomechanical characteristics as a result of the impact phase of the ground reaction force.

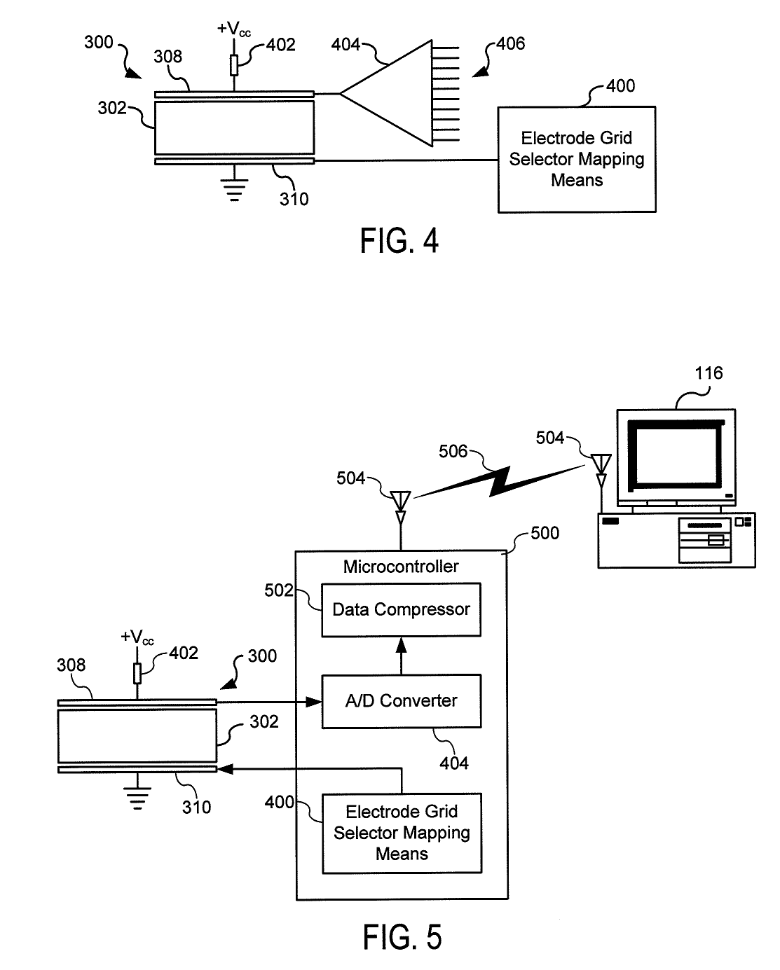

FIG. 4 illustrates in schematic format electrode grid selector mapping means 400 which may be used with the transducer shown in FIG. 3. The grid selector mapping means 400 may comprise a combination of logic, firmware, and hardware in a suitable microcontroller. Transducer 300 comprises three layers of conductive foam 302 between the electrode grids (not shown in FIG. 4) on sheets 308, 310. Such three-layer configuration is electrically coupled between +V_{cc} and ground by way of a bias resistor 402. Vfout from sheet 308 is input to a 10-bit analog-to-digital converter (ADC) 406, which outputs ten bits of digital output.

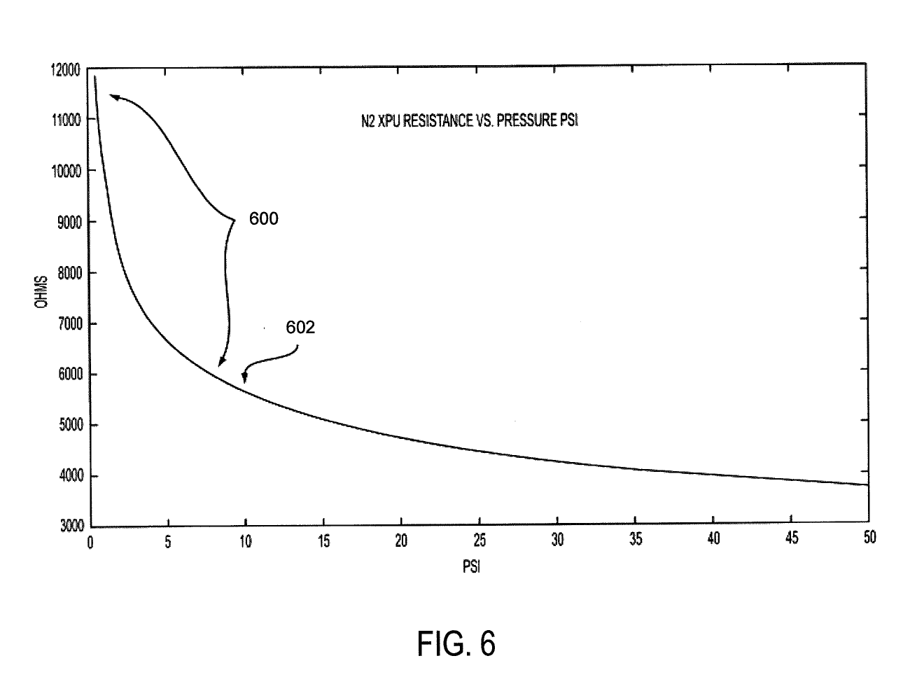

FIG. 5 illustrates in schematic format a microcontroller 500 which incorporates the electrode grid selector mapping means shown in FIG. 4.

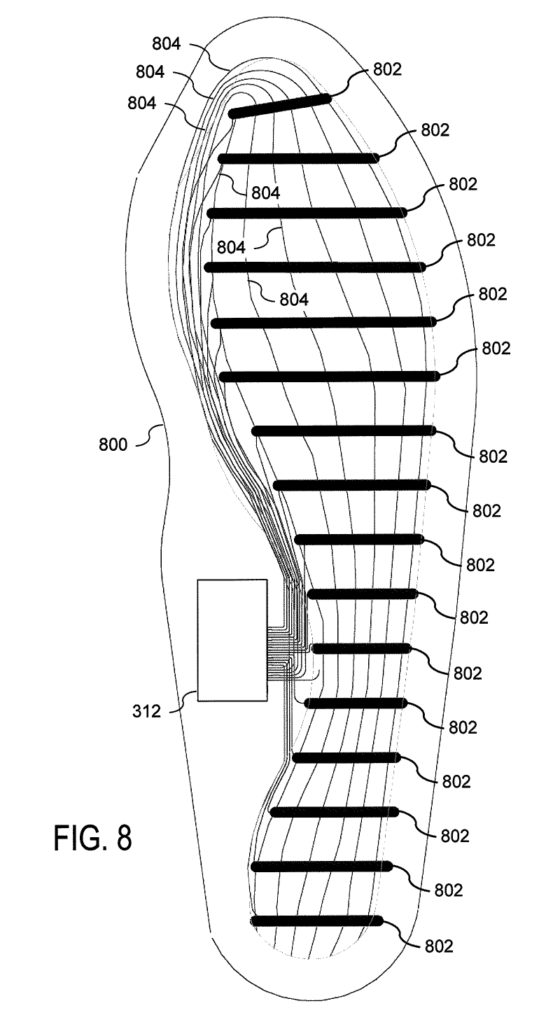

On start up, sensing system 100 first determines if it will be a collector node 114 or an insole node 106. It does this by determining first if any wired interfaces exist. This would be the case if sensing system 100 was to be a collector node 114, since a USB interface will exist to allow for attachment to computer 116. Further details regarding this process will now be described with reference to FIG. 7.

As a collector node 114, sensing system 100 would initialize the MCU, COP, GPIO, SPI, IRQ, and set the desired RF transceiver clock frequency by calling routines MCUInit, GPIOInit, SPIInit, IRQInit, IRQACK, SPIDrvRead, and IRQPinEnable at step 702. MCUInit is the master initialization routine which turns off the MCU watchdog, and sets the timer module in order to use BUSCLK as a reference with a pre-scaling of 32. The state variable gu8RTxMode would be set to SYSTEM_RESET_MODE, and routines GPIOInit, SPIInit, and IRQInit would be called. Next, the state variable gu8RTxMode would be set to RF_TRANSCEIVER_RESET_MODE and the IRQFLAG would be checked to see if IRQ is asserted. The RF transceiver interrupts would first be cleared, using SPIDrvRead. Then, RF transceiver would be checked for ATTN IRQ interrupts. As a final step for MCUInit, calls would be made to PLMEPhyReset (in order to reset the physical MAC layer), IRQACK (in order to ACK the pending IRQ interrupt), and IRQPinEnable (to pin Enable, IE, IRQ CLR, on signal's negative edge).

Once the collector node process has been initialized, sensing system 100 is ready to receive RF packets from insole nodes 106. This would be started by creating a RF packet receive queue that is driven by a call back function on RF transceiver packet receive interrupts See, e.g., step 720. When an RF packet is received from an insole node 106, a check would first be made to determine if that packet is from a new insole node 106 or an existing one. If from an existing insole node 106, RF packet sequence numbers would be checked to determine continuous synchronization before further analyzing the packet. If from a new insole node 106, an insole node context state block would be created and initialized. Above this RF packet session level process for node-to-node communication, is the analysis of the RF packet data payload. This payload would contain the compressed plantar foot pressure profile based on the current variable pressure analysis map. The first part of the compressed data would contain a map mask array, which may be structured as follows:

```
    | 0x10 |00101001|00101101|* * * * |00111101|00101010| 245 | 234 |
219 | 225 | * * * * | 233 |
    | start | row 1 | row 2 | | row 15
| row m  | D1 | D2  | D3 | D4 | |D n  |
```

where a bit in the FootMaskArray(row 1, row 2, . . . , row m) would be set to one for data that is 255 in value. Each row representation byte would use 6 bits (i.e., the upper two bits would be zero and reserved for future use) to refer to each analog-to-digital (A/D) channel, of which there are six in the current utility. Next, the FootRowMask[k] array would be scanned for non-active values (i.e., no compression). The location in the FootRowMask[k] array where to set the no compression value bit would then be determined. This may be done by first finding out which byte of 16 (which represent rows) in the FootRowMask[k] array is the row that has a no compression value in it. The base value that brings in the row byte of interest would then be removed, and the remainder may be used as a bit mask and XORed with existing contents, which could be other no compression values already identified.

Once the RF packet from an insole node 106 would be decompressed, the collector node 114 would use the SCITransmitArray routine to send such decompressed RF packet data (gsRxpacket.pu8Data and gsRxPacket.u8DataLength) to the connected computer 116 via the USB interface (not shown). The insole pressure data would then be formatted as follows:

|Packet header|0x10|value of A/D CH0|value of A/D CH1|value of A/D
CH2|value of A/D CH3|
|value of A/D CH6|value of A/D CH7|value of
A/D CH0|value of A/D CH1|
|value of A/D CH2|value of A/D CH3|value of
A/D CH6|* * * * *

The IEEE 802.15.4 standard (which will be referred to hereinafter as “802.15.4”), which is the basis for the ZigBee, WirelessHART, and MiWi specifications, specifies a maximum packet size of 127 bytes and the Time Synchronized Mesh Protocol (TSMP) reserves 47 Bytes for operation, leaving 80 Bytes for payload. The 2.4 GHz Industrial, Scientific, and Medical (ISM) band Radio Frequency (RF) transceiver which may be used herein is compliant with 802.15.4. It contains a complete 802.15.4 physical layer (PHY) modem designed for the 802.15.4 wireless standard, which supports peer-to-peer, star, and mesh networking. It is combined with an MPU to create the wireless RF data link and network according to various embodiments of the present invention. The transceiver (e.g., RF module 312) supports both 250 kbps O-QPSK data in 5.0 MHz channels and full spread-spectrum encode and decode.

All control, reading of status, writing of data, and reading of data is done through the sensing system node device's RF transceiver interface port.

The sensing system node device's MPU accesses the sensing system node device's RF transceiver through interface “transactions” in which multiple bursts of byte-long data are transmitted on the interface bus. Each transaction is three or more bursts long depending on the transaction type. Transactions are always read accesses or write accesses to register addresses. The associated data for any single register access is always 16 bits in length.

Receive mode is the state where the sensing system node device's RF transceiver is waiting for an incoming data frame. The packet receive mode allows the sensing system node device's RF transceiver to receive the whole packet without intervention from the sensing system node device's MPU. The entire packet payload may be stored in RX Packet RAM and the microcontroller fetches the data after determining the length and validity of the RX packet.

The sensing system node device's RF transceiver waits for a preamble followed by a Start of Frame Delimiter. From there, the Frame Length Indicator is used to determine the length of the frame and calculate the Cycle

Redundancy Check (CRC) sequence. After a frame is received, the sensing system node device's application determines the validity of the packet. Due to noise, it is possible for an invalid packet to be reported with either of the following conditions: a valid CRC and a frame length (0, 1, or 2) and/or an invalid CRC/invalid frame length.

The sensing system node device's application software determines if the packet CRC is valid and that the packet frame length is valid with a value of 3 or greater. In response to the interrupt request from the sensing system node device's RF transceiver, the sensing system node device's MPU determines the validity of the frame by reading and checking valid frame length and CRC data. The receive packet RAM port register is accessed when the sensing system node device's RF transceiver is read for data transfer.

The sensing system node device's RF transceiver transmits entire packets without intervention from the Invention node device's MPU. The entire packet payload is pre-loaded in TX Packet RAM, the sensing system node device's RF transceiver transmits the frame, and then the transmit complete status is set for the sensing system node device's MPU. When the packet is successfully transmitted, a transmit interrupt routine that runs on the sensing system node device's MPU reports the completion of packet transmission. In response to the interrupt request from the sensing system node device's RF transceiver, the sensing system node device's MPU reads the status to clear the interrupt and check successful transmission.

Control of the sensing system node device's RF transceiver and data transfers are accomplished by means of a Serial Peripheral Interface (SPI). Although the normal SPI protocol is based on 8-bit transfers, the sensing system node device's RF transceiver imposes a higher level transaction protocol that is based on multiple 8-bit transfers per transaction. A singular SPI read or write transaction comprises an 8-bit header transfer followed by two 8-bit data transfers. The header denotes access type and register address. The following bytes are read or write data. The SPI also supports recursive “data burst” transactions, in which additional data transfers can occur. The recursive mode is primarily intended for packet RAM access and fast configuration of the sensing system node device's RF transceiver.

When the sensing system determines that it is to operate in an insole mode, it will reset its state flag, FootstepPacketRecvd, and will call its MLMERXEnableRequest routine while enabling a LOW_POWER_WHILE state. The insole node 106 will then wait 250 milliseconds for a response from the collector node 114 to determine whether a default full insole electrode scan will be done or a mapped electrode scan will be initiated. In the case of a mapped electrode scan, the collector node send the appropriate electrode scan mapping configuration data. Electrode scanning is performed by the FootScan routine, where the FootDataBufferindex is initialized and rows are activated, by enabling MCU direction mode for output [PTCDD_PTCDDN=Output] and bringing the associated port line low[PTCD_PTCD6=0]. As each row is activated based on the electrode scanning map, the columns which are attached to the MCU analog signal ports will sample and read the current voltage on the column lines and convert them into digital form which is the plantar foot pressure across that selected row. All rows may be sequentially scanned and the entire process repeated until a reset condition or inactivity power-down mode.

The plantar foot pressure data is compressed by clearing the bit map mask array, which may be structured as follows:

| 0x10 |00101001|00101101| * * * |00111101|00101010| 245 | 234 | 219
| 225 | * * * | 233 |
|start | row 1| row 2 | * * * | row 15 | row 16 | * * * | row N
|Data1|Data2|Data3| * * * |DataN|

This is where a bit in the FootMaskArray [k] is set to one for data that is no compression in value. Each row representation byte uses 6 bits (i.e., the upper two bits would be zero and reserved for future use) to refer to each A/D channel, of which there are six. To set the compression bit, a call is made to the routine FootSetMask with parameters FootRowMaskIndex and MaskValue set accordingly. Then, based on Maskvalue, an XOR operation is performed on FootRowMask [R] with a selected mask value {0x01; 0x02; 0x04; 0x08; 0x10; 0x20;}.

Several variables such as FootSendNumBytes and FootDataBufferIndex are used to prepare 802.15.4 RF packets gsTxPacket.gau8TxDataBuffer[ ] for sending using the compressed data in FootDataBuffer[ ]. The RF packets are sent using the RFSendRequest (&gsTxPacket) routine. This routine checks to see if gu8RTxMode is set at IDLE_MODE and uses gsTxPacket as a pointer to call the RAMDrvWriteTx routine, which then calls SPIDrvRead to read the RF transceiver's TX packet length register contents. Using this contents, mask length setting may be updated with 2 added 2 for CRC and 2 for code bytes. A call is made to SPIDrvWrite to update the TX packet length field. Next, a call to SPIClearRecieveStatReg is made to clear the status register followed by a call to SpiclearRecieveDataReg to clear the receive data register to make the SPI interface ready for reading or writing.

With the SPI interface ready, a call is made to SPISendChar sending a 0xFF character which represents the 1st code byte. Next, SPIWaitTransferDone is called to verify the send is done.

Now, SPISendChar is called again to send a 0x7E byte, which is the second code byte and then the SPIwaitTransferDone is called again to verify the send is done. With these code bytes sent, the rest of the packet is sent using a for loop where psTxPkt→u8DataLength+1 are the number of iterations to a series of sequential to Spisendchar, SPIWaitTransferDone, SPIClearRecieveDataReg. Once this is done, the RF transceiver is loaded with the packet to send. The ANTENNA_SWITCH is set to transmit, the LNA_ON mode enabled and finally a RTXENAssert call made to actually send the packet.

In this manner, by using continuous two-dimensional pressure sensing grids with variable mapping capability, a three-dimensional, real-time plantar pressure may be obtained and wirelessly transmitted to a remote location for analysis and display. Further details regarding the programming of sensing system 100 in the manner described above may be found in Wireless Sensing Triple Axis Reference Design Designer Reference Manual, Document Number ZSTARRM, Rev. 3, 01/2007, and Simple Media Access Controller (SMAC) User's Guide, Document Number SMACRM, Rev. 1.2, 04/2005, each of which is a publication of Freescale Semiconductor, Inc. and is incorporated herein by reference.

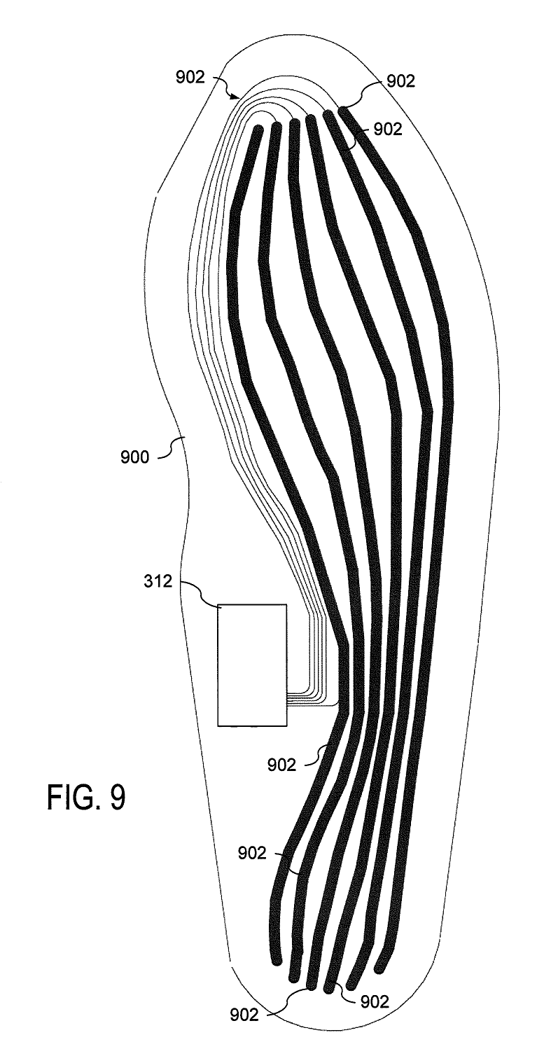

Referring now to FIG. 8, there is shown a first electrode grid layer 800 of a transducer according to another embodiment of the present invention. Layer 800 is comparable to layer 308 shown in FIG. 3. In this case, however, a plurality of lateral grid members 802 are electrically coupled to RF module 312, as are a plurality of longitudinal grid members 804.

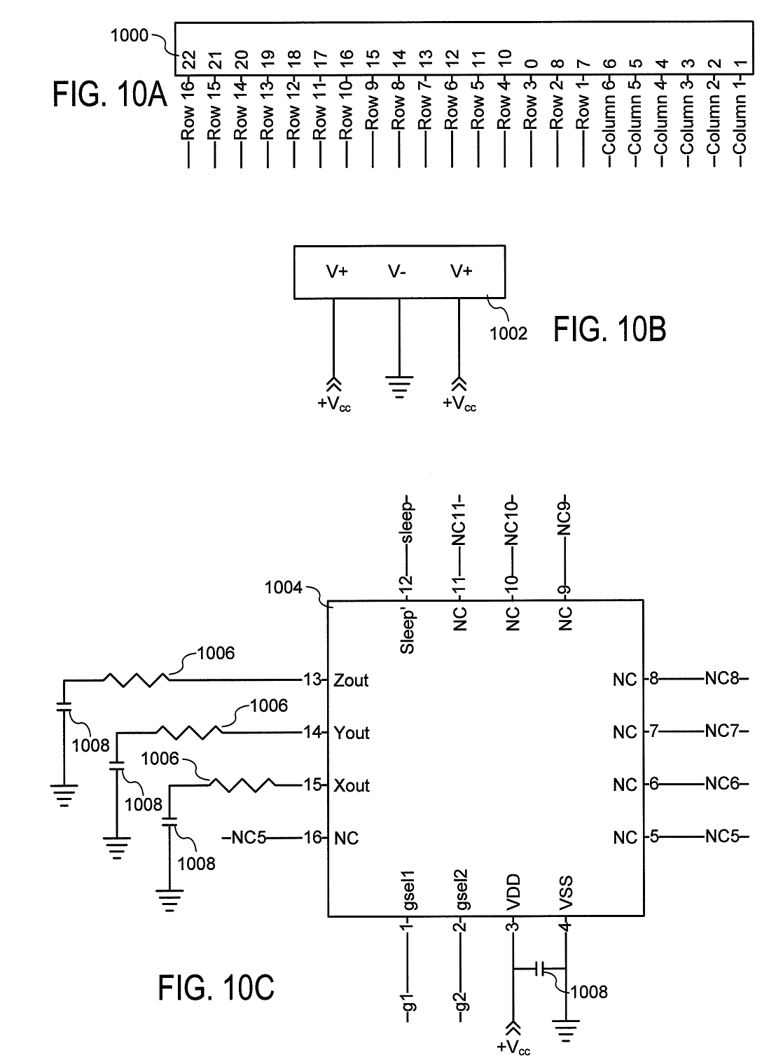

FIG. 9 illustrates a second electrode grid layer 900 which may be used with the first electrode grid layer 800. Layer 900 is comparable to layer 310 shown in FIG. 3. In this case, however, the plurality of longitudinal members 902 are coupled to RF module 312, and cooperate with the first electrode grid layer 800 and conductive foam layer 302 (not shown in FIG. 8 or 9) to sense pressure at a plurality of points of interest along the user's feet.

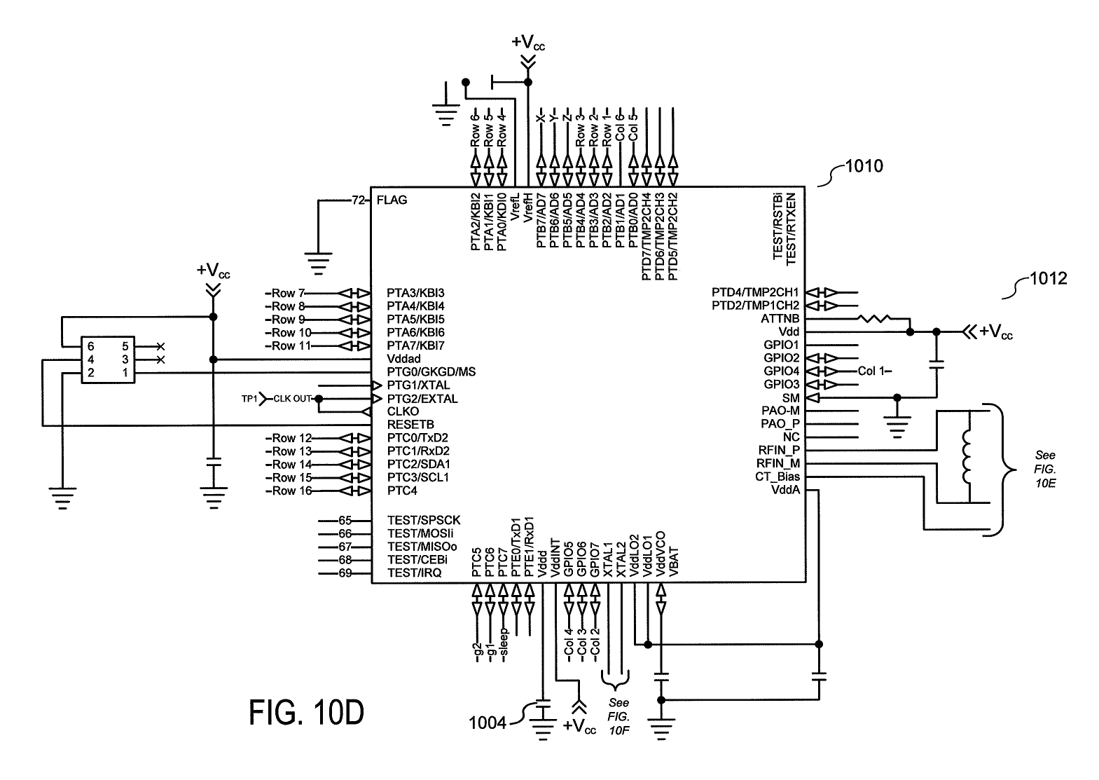

FIGS. 10A-10F illustrate in schematic format portions of an RF module 312 which may be used with the first and second electrode grid layers shown in FIGS. 8 and 9. In FIG. 10A, a portion of the schematic relating to a flexible PCB connection 1000 is shown. Rows and columns of the electronic grids in the mapped array may be coupled to the RF module 312. FIG. 10B illustrates a portion of the schematic relating to a battery 1002. A suitable battery may comprise a Model No. BK-877, with a CR2450 Coin Cell Retainer SMD made of phosphor bronze, nickel finished contacts, and a Mylar battery insulator.

Referring now to FIG. 10C, there is shown a portion of the schematic relating to a triple-axis accelerometer 1004 according to embodiments of the present invention. One exemplary accelerometer 1004 may be the model MMA726OQT ±1.5 g-6 g Three Axis Low-g Micromachined Accelerometer manufactured by Freescale Semiconductor, Inc. of Tempe, Ariz. USA. This low cost capacitive micromachined accelerometer features signal conditioning, a 1-pole low pass filter, temperature compensation and g-Select which allows for the selection among 4 sensitivities. Zero-g offset full scale span and filter cut-off are factory set and require no external devices. It includes a Sleep Mode that makes it ideal for handheld battery powered electronics.

Accelerometer 1004 is a surface-micromachined integrated-circuit accelerometer. The device consists of two surface micromachined capacitive sensing cells (g-cell) and a signal conditioning ASIC contained in a single integrated circuit package. The sensing elements are sealed hermetically at the wafer level using a bulk micromachined cap wafer.

The g-cell is a mechanical structure formed from semiconductor materials (polysilicon) using semiconductor processes (masking and etching). It can be modeled as a set of beams attached to a movable central mass that move between fixed beams. The movable beams can be deflected from their rest position by subjecting the system to an acceleration.

As the beams attached to the central mass move, the distance from them to the fixed beams on one side will increase by the same amount that the distance to the fixed beams on the other side decreases. The change in distance is a measure of acceleration.

The g-cell beams form two back-to-back capacitors. As the center beam moves with acceleration, the distance between the beams changes and each capacitor's value will change, (C=Aε/D). Where A is the area of the beam, ε is the dielectric constant, and D is the distance between the beams.

The ASIC uses switched capacitor techniques to measure the g-cell capacitors and extract the acceleration data from the difference between the two capacitors. The ASIC also signal conditions and filters (switched capacitor) the signal, providing a high level output voltage that is ratiometric and proportional to acceleration.

The g-Select feature allows for the selection among 4 sensitivities present in the device. Depending on the logic input placed on pins 1 and 2, the device internal gain will be changed allowing it to function with a 1.5 g, 2 g, 4 g, or 6 g sensitivity (Table 1 below). This feature is ideal when a product has applications requiring different sensitivities for optimum performance. The sensitivity can be changed at anytime during the operation of the product. The g-Select1 and g-Select2 pins can be left unconnected for applications requiring only a 1.5 g sensitivity as the device has an internal pull-down to keep it at that sensitivity (800 mV/g).

TABLE 1
g-Select Pin Descriptions

| g-Select2 | g-Select1 | g-Range | Sensitivity |
|---|---|---|---|
| 0 | 0 | 1.5 g | 800 mV/g |
| 0 | 1 | 2 g | 600 mV/g |
| 1 | 0 | 4 g | 300 mV/g |
| 1 | 1 | 6 g | 200 mV/g |

Accelerometer 1004 may provide a Sleep Mode that is ideal for battery operated products. When Sleep Mode is active, the device outputs are turned off, providing significant reduction of operating current. A low input signal on pin 12 (Sleep Mode) will place the device in this mode and reduce the current to 3 μA typ. For lower power consumption, it is recommended to set g-Select1 and g-Select2 to 1.5 g mode. By placing a high input signal on pin 12, the device will resume to normal mode of operation.

Accelerometer 1004 also contains onboard single-pole switched capacitor filters. Because the filter is realized using switched capacitor techniques, there is no requirement for external passive components (i.e., resistors and capacitors) to set the cut-off frequency.

Ratiometricity simply means the output offset voltage and sensitivity will scale linearly with applied supply voltage. That is, as supply voltage is increased, the sensitivity and offset increase linearly; as supply voltage decreases, offset and sensitivity decrease linearly. This is a key feature when interfacing to a microcontroller or an A/D converter because it provides system level cancellation of supply induced errors in the analog to digital conversion process. Offset ratiometric error can be typically >20% at VDD=2.2 V. Sensitivity ratiometric error can be typically >3% at VDD=2.2 V.

TABLE 2
Pin Descriptions

| Pin No. | Pin Name | Description |
|---|---|---|
| 1 | g-Select1 | Logic input pin to select g level. |
| 2 | g-Select2 | Logic input pin to select g level. |
| 3 | VDD | Power Supply Input |
| 4 | VSS | Power Supply Ground |
| 5-7 | N/C | No internal connection. Leave unconnected. |
| 8-11 | N/C | Unused for factory trim. Leave unconnected. |
| 12 | Sleep Mode | Logic input pin to enable product or Sleep Mode. |
| 13 | ZOUT | Z direction output voltage. |
| 14 | YOUT | Y direction output voltage. |
| 15 | XOUT | X direction output voltage. |
| 16 | N/C | No internal connection. Leave unconnected. |

The VDD line should have the ability to reach 2.2 V in <0.1 ms as measured on the device at the VDD pin. Rise times greater than this most likely will prevent start up operation. Physical coupling distance of the accelerometer to the microcontroller should be minimal. The flag underneath the package is internally connected to ground. It is not recommended for the flag to be soldered down. A ground plane should be placed beneath the accelerometer 1004 to reduce noise. The ground plane should be attached to all of the open ended terminals. An RC filter with a 1.0 kΩ resistor 1006 and a 0.1 μF capacitor 1008 may be used on the outputs of the accelerometer 1004 in order to minimize clock noise (from the switched capacitor filter circuit). PCB layout of power and ground should not couple power supply noise. Accelerometer and microcontroller should not be a high current path. A/D sampling rate and any external power supply switching frequency should be selected such that they do not interfere with the internal accelerometer sampling frequency (11 kHz for the sampling frequency). This will prevent aliasing errors. PCB layout should not run traces or vias under the QFN part. This could lead to ground shorting to the accelerometer flag.

Further details regarding accelerometer 1004 may be found in Freescale Document Number: MMA7260QT, Rev 5, 03/2008, which is incorporated herein by reference.

Referring now to FIGS. 10D-10F, there are shown portions of the schematic relating to a microcontroller 1010 and transceiver 1012, including a balun 1014 and crystal oscillator 1016, which may be used according to embodiments of the present invention. One exemplary platform incorporating both functions may be the model MC13213 ZigBee™—Compliant Platform—2.4 GHz Low Power Transceiver for the IEEE® 802.15.4 Standard plus Microcontroller manufactured by Freescale Semiconductor, Inc. of Tempe, Ariz. USA.

The MC1321x family is Freescale's second-generation ZigBee platform which incorporates a low power 2.4 GHz radio frequency transceiver and an 8-bit microcontroller into a single 9×9×1 mm 71-pin LGA package. The MC1321x solution can be used for wireless applications from simple proprietary point-to-point connectivity to a complete ZigBee mesh network. The combination of the radio and a microcontroller in a small footprint package allows for a cost-effective solution.

The MC1321x contains an RF transceiver which is an 802.15.4 compliant radio that operates in the 2.4 GHz ISM frequency band. The transceiver includes a low noise amplifier, 1 mW nominal output power, PA with internal voltage controlled oscillator (VCO), integrated transmit/receive switch, on-board power supply regulation, and full spread-spectrum encoding and decoding. The MC1321x also contains a microcontroller based on the HCS08 Family of Microcontroller Units (MCU), specifically the HCS08 Version A, and can provide up to 60 KB of flash memory and 4 KB of RAM. The onboard MCU allows the communications stack and also the application to reside on the same system-in-package (SIP). The MC13213 contains 60K of flash and 4 KB of RAM and is also intended for use with the Freescale fully compliant 802.15.4 MAC and the fully ZigBee compliant Freescale BeeStack.

TABLE 3
Pin Function Description

| Pin # | Pin Name | Type | Description | Functionality |
|---|---|---|---|---|
| 1 | PTA3/KBI1P3 | Digital Input/Output | MCU Port A Bit | |
| | | | 3/Keyboard Input | |
| | | | Bit 3 | |
| 2 | PTA4/KBI1P4 | Digital Input/Output | MCU Port A Bit | |
| | | | 4/Keyboard Input | |
| | | | Bit 4 | |
| 3 | PTA5/KBI1P5 | Digital Input/Output | MCU Port A Bit | |
| | | | 5/Keyboard Input | |
| | | | Bit 5 | |
| 4 | PTA6/KBI1P6 | Digital Input/Output | MCU Port A Bit | |
| | | | 6/Keyboard Input | |
| | | | Bit 6 | |
| 5 | PTA7/KBI1P7 | Digital Input/Output | MCU Port A Bit | |
| | | | 7/Keyboard Input | |
| | | | Bit 7 | |
| 6 | VDDAD | Power Input | MCU power | Decouple to ground. |
| | | | supply to ATD | |
| 7 | PTG0/BKGND/MS | Digital Input/Output | MCU Port G Bit | PTG0 is output only. |
| | | | 0/Background/ | Pin is I/O when used |
| | | | Mode Select | as BDM function. |
| 8 | PTG1/XTAL | Digital | MCU Port G Bit | Full I/O when not used |
| | | Input/Output/Output | 1/Crystal | as clock source. |
| | | | oscillator output | |
| 9 | PTG2/EXTAL | Digital | MCU Port G Bit | Full I/O when not used |
| | | Input/Output/Input | 2/Crystal | as clock source. |
| | | | oscillator input | |
| 10 | CLKO | Digital Output | Modem Clock | Programmable |
| | | | Output | frequencies of: 16 MHz, |
| | | | | 8 MHz, 4 MHz, 2 MHz, |
| | | | | 1 MHz, 62.5 kHz, |
| | | | | 32.786+ kHz |
| | | | | (default), and 16.393+ kHz. |
| 11 | RESET | Digital Input/Output | MCU reset. | |
| | | | Active low | |
| 12 | PTC0/TXD2 | Digital Input/Output | MCU Port C Bit | |
| | | | 0/SCI2 TX data | |
| | | | out | |
| 13 | PTC1/RXD2 | Digital Input/Output | MCU Port C Bit | |
| | | | 1/SCI2 RX data | |
| | | | in | |
| 14 | PTC2/SDA1 | Digital Input/Output | MCU Port C Bit | |
| | | | 1/IIC bus data | |
| 15 | PTC3/SCL1 | Digital Input/Output | MCU Port C Bit | |
| | | | 1/IIC bus clock | |
| 16 | PTC4 | Digital Input/Output | MCU Port C Bit 4 | |
| 17 | PTC5 | Digital Input/Output | MCU Port C Bit 5 | |
| 18 | PTC6 | Digital Input/Output | MCU Port C Bit 6 | |
| 19 | PTC7 | Digital Input/Output | MCU Port C Bit 7 | |
| 20 | PTE0/TXD1 | Digital Input/Output | MCU Port E Bit 0/ | |
| | | | SCI1 TX data | |
| | | | out | |
| 21 | PTE1/RXD1 | Digital Input/Output | MCU Port E Bit | |
| | | | 1/SCI1 RX data | |
| | | | in | |
| 22 | VDDD | Power Output | Modem regulated | Decouple to ground. |
| | | | output supply | |
| | | | voltage | |
| 23 | VDDINT | Power Input | Modem digital | 2.0 to 3.4 V. Decouple |
| | | | interface supply | to ground. Connect to |
| | | | | Battery. |
| 24 | GPIO51 | Digital Input/Output | General Purpose | See Footnote 1 |
| | | | Input/Output 5. | |
| 25 | GPIO61 | Digital Input/Output | Modem General | See Footnote 1 |
| | | | Purpose | |
| | | | Input/Output 6 | |
| 26 | GPIO71 | Digital Input/Output | Modem General | See Footnote 1 |
| | | | Purpose | |
| | | | Input/Output 7 | |
| 27 | XTAL1 | Input | Modem crystal | Connect to 16 MHz |
| | | | reference | crystal and load |
| | | | oscillator input | capacitor. |
| 28 | XTAL2 | Input/Output | Modem crystal | Connect to 16 MHz |
| | | | reference | crystal and load |
| | | | oscillator output | capacitor. Do not load |
| | | | | this pin by using it as a |
| | | | | 16 MHz source. |
| | | | | Measure 16 MHz |
| | | | | output at CLKO, |
| | | | | programmed for 16 MHz. |
| 29 | VDDLO2 | Power Input | Modem LO2 | Connect to VDDA |
| | | | VDD supply | externally. |
| 30 | VDDLO1 | Power Input | Modem LO1 | Connect to VDDA |
| | | | VDD supply | externally. |
| 31 | VDDVCO | Power Output | Modem VCO | Decouple to ground. |
| | | | regulated supply | |
| | | | bypass | |
| 32 | VBATT | Power Input | Modem voltage | Decouple to ground. |
| | | | regulators' input | Connect to Battery. |
| 33 | VDDA | Power Output | Modem analog | Decouple to ground. |
| | | | regulated supply | Connect to directly |
| | | | output | VDDLO1 and |
| | | | | VDDLO2 externally |
| | | | | and to PAO_P and |
| | | | | PAO_M through a bias |
| | | | | network. |
| 34 | CT | Bias RF Control | Modem bias | When used with |
| | | Output | voltage/control | internal T/R switch, |
| | | | signal for RF | provides ground |
| | | | external | reference for RX and |
| | | | components | VDDA reference for |
| | | | | TX. Can also be used |
| | | | | as a control signal with |
| | | | | external LNA, antenna |
| | | | | switch, and/or PA (high |
| | | | | level is VDDA). |
| 35 | RFIN_M | RF Input (Output) | Modem RF | When used with |
| | | | input/output | internal T/R switch, this |
| | | | negative | is a bi-directional RF |
| | | | | port for the internal |
| | | | | LNA and PA |
| 36 | RFIN_P | RF Input (Output) | Modem RF | When used with |
| | | | input/output | internal T/R switch, this |
| | | | positive | is a bi-directional RF |
| | | | | port for the internal |
| | | | | LNA and PA |
| 37 | NC | Not used | | May be grounded or |
| | | | | left open |
| 38 | PAO_P | RF Output | Modem power | Open drain. Connect |
| | | | amplifier RF | to VDDA through a |
| | | | output positive | bias network when |
| | | | | used with external |
| | | | | balun. Not used when |
| | | | | internal T/R switch is |
| | | | | used. |
| 39 | PAO_M | RF Output | Modem power | Open drain. Connect |
| | | | amplifier RF | to VDDA through a |
| | | | output negative | bias network when |
| | | | | used with external |
| | | | | balun. Not used when |
| | | | | internal T/R switch is |
| | | | | used. |
| 40 | SM | Input | Test Mode pin | Must be grounded for |
| | | | | normal operation |
| 41 | GPIO41 | Digital Input/Output | General Purpose | See Footnote 1 |
| | | | Input/Output 4. | |
| 42 | GPIO31 | Digital Input/Output | Modem General | See Footnote 1 |
| | | | Purpose | |
| | | | Input/Output 3 | |
| 43 | GPIO2 | Test Point | MCU Port E Bit | Internally connected |
| | | | 6/Modem | pins. When |
| | | | General Purpose | gpio_alt_en, Register |
| | | | Input/Output 2 | 9, Bit 7 = 1, GPIO2 |
| | | | | functions as a “CRC |
| | | | | Valid” indicator. |
| 44 | GPIO1 | Test Point | MCU Port E Bit | Internally connected |
| | | | 7/Modem | pins. When |
| | | | General Purpose | gpio_alt_en, Register |
| | | | Input/Output 1 | 9, Bit 7 = 1, GPIO1 |
| | | | | functions as an “Out of |
| | | | | Idle” indicator. |
| 45 | VDD | Power Input | MCU main power | Decouple to ground. |
| | | | supply | |
| 46 | ATTN2 | Digital Input | Active Low | See Footnote 2 |
| | | | Attention. | |
| | | | Transitions IC | |
| | | | from either | |
| | | | Hibernate or | |
| | | | Doze Modes to | |
| | | | Idle. | |
| 47 | PTD2/TPM1CH2 | Digital Input/Output | MCU Port D Bit | |
| | | | 2/TPM1 Channel 2 | |
| 48 | PTD4/TPM2CH1 | Digital Input/Output | MCU Port D Bit | |
| | | | 4/TPM2 Channel 1 | |
| 49 | PTD5/TPM2CH2 | Digital Input/Output | MCU Port D Bit | |
| | | | 5/TPM2 Channel 2 | |
| 50 | PTD6/TPM2CH3 | Digital Input/Output | MCU Port D Bit | |
| | | | 6/TPM2 Channel 3 | |
| 51 | PTD7/TPM2CH4 | Digital Input/Output | MCU Port D Bit | |
| | | | 7/TPM2 Channel 4 | |
| 52 | PTB0/AD1P0 | Input/Output | MCU Port B Bit | |
| | | | 0/ATD | |
| | | | analog Channel 0 | |
| 53 | PTB1/AD1P1 | Input/Output | MCU Port B Bit | |
| | | | 1/ATD analog | |
| | | | Channel 1 | |
| 54 | PTB2/AD1P2 | Input/Output | MCU Port B Bit | |
| | | | 2/ATD analog | |
| | | | Channel 2 | |
| 55 | PTB3/AD1P3 | Input/Output | MCU Port B Bit | |
| | | | 3/ATD analog | |
| | | | Channel 3 | |
| 56 | PTB4/AD1P4 | Input/Output | MCU Port B Bit | |
| | | | 4/ATD analog | |
| | | | Channel 4 | |
| 57 | PTB5/AD1P5 | Input/Output | MCU Port B Bit | |
| | | | 5/ATD analog | |
| | | | Channel 5 | |
| 58 | PTB6/AD1P6 | Input/Output | MCU Port B Bit | |
| | | | 6/ATD analog | |
| | | | Channel 6 | |
| 59 | PTB7/AD1P7 | Input/Output | MCU Port B Bit | |
| | | | 7/ATD analog | |
| | | | Channel 7 | |
| 60 | VREFH | Input | MCU high | |
| | | | reference voltage | |
| | | | for ATD | |
| 61 | VREFL | Input | MCU low | |
| | | | reference voltage | |
| | | | for ATD | |
| 62 | PTA0/KBI1P0 | Digital Input/Output | MCU Port A Bit | |
| | | | 0/Keyboard Input | |
| | | | Bit 0 | |
| 63 | PTA1/KBI1P1 | Digital Input/Output | MCU Port A Bit | |
| | | | 1/Keyboard Input | |
| | | | Bit 1 | |
| 64 | PTA2/KBI1P2 | Digital Input/Output | MCU Port A Bit | |
| | | | 2/Keyboard Input | |
| | | | Bit 2 | |
| 65 | TEST | Test Point | For factory test | Do not connect |
| 66 | TEST | Test Point | For factory test | Do not connect |
| 67 | TEST | Test Point | For factory test | Do not connect |
| 68 | TEST | Test Point | For factory test | Do not connect |
| 69 | TEST | Test Point | For factory test | Do not connect |
| 70 | TEST | Test Point | For factory test | Do not connect |
| 71 | TEST | Test Point | For factory test | Do not connect |
| FLAG | VSS | Power input | External package | Connect to ground. |
| | | | flag. Common | |
| | | | VSS | |

¹The transceiver GPIO pins default to inputs at reset. There are no programmable pull ups on these pins. Unused GPIO pins should be tied to ground if left as inputs, or if left unconnected, they should be programmed as outputs set to the low state.
²During low power modes, input must remain driven by MCU.

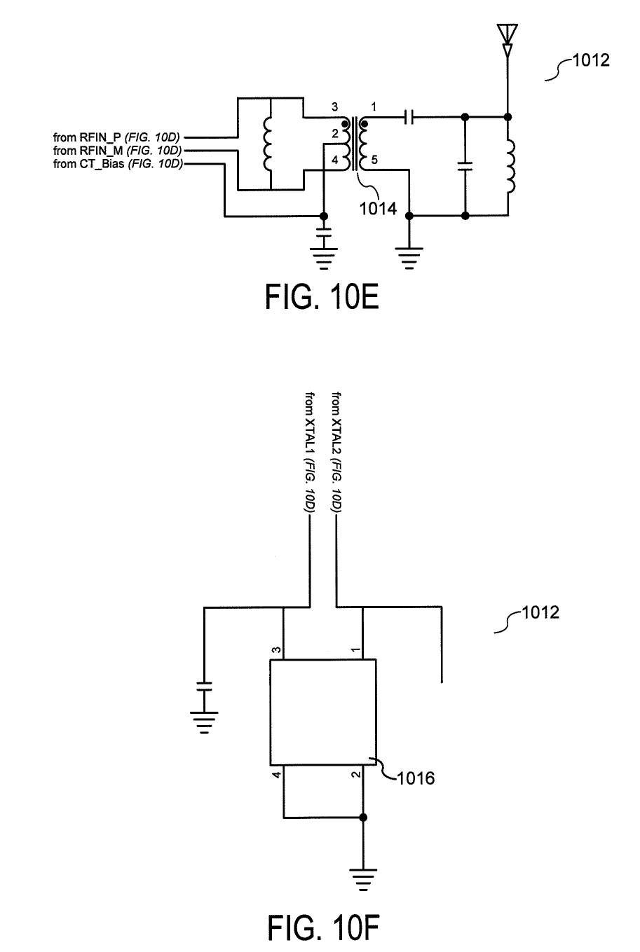

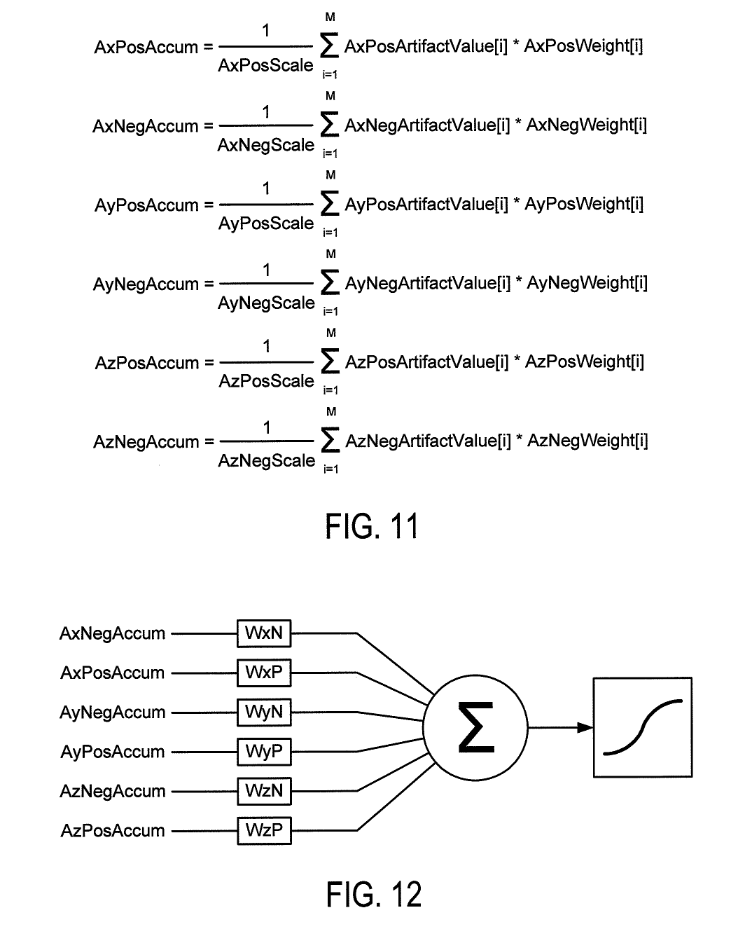

FIGS. 11 and 12 illustrate a first and a second algorithm which may be used with transducers according to FIGS. 8, 9 and 10A-10F. Sensing system 100 may use an exponential moving average filter in conjunction with a sliding window boxcar style integrator to per-process digitize real-time acceleration data for all three dimensions Ax, Ay, Az. The accumulated acceleration data may be analyzed to identify unique motion artifacts such as strides and steps and their respective directions. Reference frames may be created to provide variable time sequences of motion artifacts. The XPU conductive foam allows for gating reference frames such as the start of step (i.e., a standing position—rising foot to start stride) and the end of step (i.e., a falling foot—to standing position). A general algorithm which may be incorporated and implemented as embedded software running on processors supporting the sensing system 100 is as follows:

$$A ( x , y , z ) SigAccum = \frac{1}{\begin{matrix}A ( x , y , z ) \\ SigScale\end{matrix}} * {\sum }_{i = 1}^{M} \vert A ( x , y , z ) [ i ] \vert * Aw ( x , y , z ) [ i ]$$

Alternative embodiments of that algorithm are shown in FIG. 11. In either case, the results of those algorithms are summed and integrated in the manner shown in FIG. 12. The resultant approximates the following:

$$M ( \sim Ax , \sim Ay , \sim Az , t ) = [ \begin{matrix}AxNegAccum * WxN + \\ AxPosAccum * WxP + \\ AyNegAccum * WyN + \\ AyPosAccum * WyP + \\ AzNegAccum * WzN + \\ AzPosAccum * WzP\end{matrix} ] * [ 1 + exp ( - t ) ] .$$

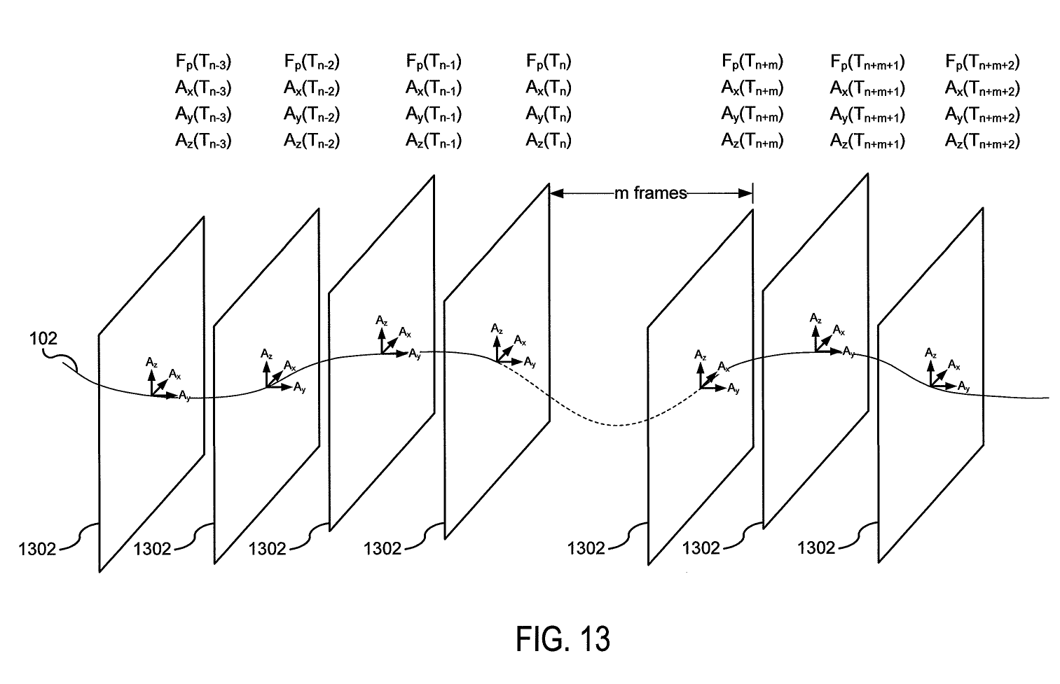

FIG. 13 illustrates an insole data capture process using the algorithms shown in FIGS. 11 and 12. Foot pressure and motion data are captured as a series of frames 1302, which occur at 128 times per second for each foot utilizing an insole 102 (FIG. 1). In this illustration, the time sequence starts at time Tₙ₋₃ and stops at time Tₙ₊ₘ₊₂.

Sensing system 100 is, thus, sensitive enough to measure the plantar pressures differences between adult male and female foot pressures under the longitudinal arch. Under the mid-foot, females have reduced peak foot pressures during standing. Also, for females, there is a correlation between body weight and foot pressures under the longitudinal arch of a female's feet in walking. This allows for sensing system 100 to analyze the ligamentous structure which results to some degree in collapse of the longitudinal arch during weight bearing phase of walking.

Sensing system 100 is able to perform similar foot function analysis during running. Specifically, sensing system 100 may analyze mid-foot loading as well as the amount of rear-foot rotation which is more apparent in female runners as compared to male runners. In the case for children, contrary to adults, body weight is identified to be of major influence on the magnitude of the pressures under the feet of children and between boys and girls no differences in the foot pressure or relative load patterns are present. Sensing system 100 may be used in such cases periodically to analyze potential walking/running/gait related issues in children as they develop. This may provide data that may help in development of proper in-soles and other support structures to aid in the renormalizing walking/running/gait related issues.

Sensing system 100 may also help determine the cause of pain and lower extremity complaints for overweight and obese persons. The system's ability to analyze plantar pressure analysis may provide additional insight into pain and lower extremity complaints. Plantar pressure differences between obese and non-obese adults during standing and walking indicates that the overweight persons have an increase in the forefoot width to foot length ratio. This is due to the broadening of the forefoot under increased weight loading conditions. Even though there is the increased load bearing contact area with the foot against the ground, overweight persons have substantially higher foot pressures under the heel, mid-foot, and forefoot during standing, walking and running.

Sensing system 100 measures larger foot pressures under the mid-foot during standing periods for the obese women as compared to the obese men. There is a major influence of body weight on the flattening of the arch is the consequence of the inherent reduced strength of the ligaments in natively in women's feet. This may contribute to lower extremity pain and discomfort in these obese persons and their choice of footwear and predisposition to participation in activities of daily living such as walking and running. For walking, the forefoot pressures as well as the forefoot contact area are substantially increased for obese women. Sensing system 100 may analyze and monitor this increased forefoot plantar pressures, which in most cases result in foot discomfort and hinders these obese women in participating normally in physical activity.

Sensing system 100 may also help runners manage overuse injuries. This affects more than half of active runners each year and causes them to stop running. The causes of such injuries include variation/distribution of body dimensions to optimize training, rear-foot movement, kinetic, and strength variables. Biomechanical parameters such as real-time foot pressures may be identified and analyzed by sensing system 100 to help identify key properties of athletic footwear in providing overuse injury protection and performance enhancement. Such parameters may be mid-sole material properties, which may provide information about footwear production tolerances.

Sensing system 100 may also measure and record rear-foot rotation, foot pressure patterns, and shock absorption properties running shoes/athletic footwear to analyze shoe characteristics which may help reduce the risk of overuse injuries. Thus, sensing system 100 may be used to evaluate shoe fit and comfort during running on various terrain types. The system's long term monitoring and archive capability allows for analyzing deterioration of shoe properties over time and use.

Sensing system 100 may also record in real-time in-shoe pressure during running and training and provides information of the interaction between footwear and foot mechanics of the person wearing them. Over rotation during running and training is responsible for many overuse injuries. Typically, restriction of excessive rear-foot motion and improved shock absorption may reduce the risk of running and training injuries. The determination and measurement of subtalar joint rotation are critical the evaluation of running and training shoes. Capturing real-time subtalar joint rotation measurement data is one of the main features of the sensing system.

Sensing system 100 may also determine wear and tear with the assessment monitoring and recording features. It has the ability to detect, capture and analyze foot pressure data wirelessly and in real-time variations in rear-foot motion combined with the differences in mid-sole properties to determine shoe cushioning differences to categorize overall stiffness of the shoe. These stiffness characteristic tend to alter the wears landing patterns to elicit lower impact forces. This allows for constructing biomechanical assessments that are beneficial for the wearer using such shoes to minimize injuries resulting from repeated impact loading. The wear of the insole will be displayed outside the shoe as green, yellow, red graphic display indications to illustrate the degree of shoe wear.

Sensing system 100 may also perform weight and power assessment by foot zones (e.g., heel, mid-foot, and forefoot). Sensing system 100 has capability to detect, capture and analyze foot pressure data wirelessly and in real-time relating to vertical ground reaction force patterns and materials characterization of running shoes with advanced cushioning column systems during walking, running, and/or training.

Sensing system 100 may also detect changes in foot sole pressure patterns during activity so that a subject's footfall changes/patterns may be determined during a specific event and correlated against multiple events (e.g., practice versus game activity). To be able to detect slight variations of pressure over time—like the loss of fluid within a running race. The ability to transmit this information wirelessly to a collection site or monitor.

Sensing system 100 may also detect changes in power patterns during a specific sporting event and calculate power/energy requirements against expected output. Energy vector analysis versus current and expected output.

Sensing system 100 may also provide the monitoring and analysis required for dance and kinesiology applications, interactive dance movements (e.g., learn to dance as a game application where a subject is signaled in one way when they are taking the right steps and another when they are wrong.

Sensing system 100 may also provide the monitoring and analysis required for industrial applications to determine warehouse personnel effectiveness, such as allowable personnel movements measured against assembly efficiency, the determination of specific individuals locations (since GPS is not very effective and expensive to deploy indoors, especially in a warehouse setting), to guard against entry into certain areas where they are prohibited such as hazard and/or security areas, and in applications where there are employee health care incentives for weight loss and health maintenance.

Sensing system 100 may also may augment gaming interfaces to supplement videogames such as PlayStation PS3 and XBox 360 gaming console. This would add an extra dimension to how one interacts with videogames running on these game consoles. Foot pressure activity detected during jumping, walking or running are combined with foot orientation and location data to provide enhance interactivity to the regular popular videogames, allowing for intuitive game play such as kicking or blocking in a fighting game.

A backend server processing option of sensing system 100 may also be able to collect large groups of insole monitors that would represent a field of players involved in sporting games (e.g., football, soccer, basketball and the like). This may be implemented as a website for remote analysis supporting peer review type applications. Sensing system 100 may also be able to capture the data over a large field of reference (e.g., sports field, field of battle, long distance run) by a specific signature for an individual sole, by person (i.e., two soles) or by collection of individuals. Sensing system 100 would, thus, enable download of all of this information upon arrival, within a transmission zone, to a web interface that creates a post event re-simulation to be stored, compared and rated by peer web gamers.

The backend server processing option is also able to collect large groups of the insole monitors that would represent a field of players involved in sporting games (e.g., football, soccer, basketball, and the like). This may allow for the creation of game strategy analysis program by using correlation analysis using real-time and archived in-sole data. With additional data input, such as real-time video, it would be readily apparent to those of ordinary skill in the art that enhanced dynamic game strategy adjustment programs would be possible.

Sensing system 100 may also be able to detect slight variations of foot pressure over time caused by conditions such as the loss of fluid within a running race, the change in pressure in a medical or rehabilitation environment, the change in pressure during an operating process (e.g., driving a car) where pressure may indicate that the operator is fit to continue. With the monitoring and archive capabilities of sensing system 100, programs may be constructed to manage long-term foot pressure variation analysis as previously mentioned.

Sensing system 100 may also be implemented in a floor mat type arrangement for a car as the key mechanism for vehicle speed operation. It may also be used in applications to assist in small motor control where the operator is incapable, either due to injury or birth defect, of applying pressure to hand or foot operating systems. In both cases mentioned, wireless support for sensing system 100 allows for six-degrees of motion.

Yet another embodiment of sensing system 100 is one in which energy is “harvested”. That is, piezoelectric fiber composites can convert mechanical energy and into electrical energy. Alternative embodiments of sensing system 100 may be used to leverage the composite nature of such piezoelectric fiber composites, because they are lighter and more flexible than bulk piezoelectric ceramics. Such piezoelectric fiber composites are capable of producing 50 V at a “stepping” frequency of 3 Hz. This could charge a battery at a 5 milliamp rate. Piezoelectric fiber composites may be shaped within insoles of various embodiments of the present invention, running from heel to toe. Piezoelectric fiber composites may also run in parallel, to accumulate the desired electric power. As a result, sensing system 100 may leverage potted and laminated implementations in conjunction with polyethylene sheets for insole design.

Such sensing systems 100, including piezoelectric fiber composites would be very durable and have a fatigue life time which is greater than 200 million cycles, with no degradation in the piezoelectric characteristics. The piezoelectric fiber composites used herein are 250 microns in diameter with variable lengths. A charging circuit could be added to provide voltage limiting and conditioning capabilities for a battery charging application. The particular battery technology which would be useful for sensing system 100 would be a function of its application. For example, gaming, sports and health monitoring applications might require a rechargeable Lithium-Polymer (Li-Poly) battery. In such cases, a 1 mm insole layer of piezoelectric fiber composites would be appropriate for battery recharging implementations.

Alternative Embodiments of Sensing System 100

Another form of garment which may incorporate sensing system 100 is a handgrip device, which has the ability to capture both handgrip strength and hand motion data at the same time. The handgrip data may then be analyzed and archived in real-time into any available computer with a standard USB-type connection. A specific set of applications utilizing such a handgrip device may be used to measure handgrip strength and hand mobility over designated time periods for the purposes of determining a progression of certain diseases and potential medication scheduling issues.

A series of analytics supporting the handgrip device may also be used to allow for correlation studies as one of the baseline features. This will allow for evaluating a person's upper extremity muscle strength and bone mass. A reduction in handgrip strength, for example, may indicate fatigue, pain, and other factors. Handgrip strength in persons with fibromyalgia syndrome (FMS) is a direct indicator of physical function, pain severity, and quality of life. Persons with FMS have lower maximal respiratory pressures than healthy people, indicating reduced pulmonary muscle strength. There is a direct correlation in handgrip strength versus non-respiratory skeletal muscle force, which indicates reduced pulmonary muscle strength. This reduction in peripheral skeletal muscle performance is measured via handgrip strength.

The decline in handgrip strength in older persons can be associated with an increased risk of Alzheimer's disease. Handgrip strength, together with three-dimensional motion analysis, allows for the evaluation of the motor activities of limbs in patients suffering from Parkinson's disease. The captured biomedical data can identify and characterize the motor activities of limbs. The study of these parameters and the analysis of the correlations between this acquired data permits mining useful information and details about the objective evaluation of Parkinson pathology.

In the evaluation of chronic liver disease (CLD), the assessment of muscle function, specifically handgrip strength, is also an important tool. These evaluation techniques may also be used for people with amyotrophic lateral sclerosis (ALS). The requirements for such evaluation techniques are: low test-retest variations, archive mild/severe impairment episodes, time-efficient, inexpensive, sensitive to small changes in measure, easy to learn, support data-warehousing, provide correlation analysis and support multi-institutional studies.

Handgrips may also be used as a game control device to measure the player/participant's grip pressure as an augmentation to existing game controller functions such as buttons and switches that control the game. Other smaller versions of such handgrip devices incorporating sensing system 100 may be used, for example, on newborns to identify early developmental issues; to augment automobile and airplane steering and guidance systems; and to detect reduced operator effectiveness or proficiency with paraplegic patients.

Yet another form of garment which may incorporate sensing system 100 is a wireless glove. Various sensor technologies may be used to capture physical data, such as hand movement and bending of the fingers. Accelerometers and pressure sensors may be attached to capture the global position data and finger pressure data of such as glove. These movements and finger pressure are then interpreted by the software that accompanies the glove, so any one movement can mean any number of things. Gesture analysis can also be performed can then be categorized into basic motion information groups, such as to recognize sign language or other group or symbolic functions.

A glove-based form factor of sensing system 100 (or “wireless glove”) may also be used to provide haptic feedback. Haptic feedback, often referred to as simply “haptics”, is the use of the sense of touch in a user interface design to provide information to an end user. For example, when used in reference to mobile phones and similar devices, this generally means the use of vibrations from the device's vibration alarm to denote that a touchscreen button has been pressed. In this particular example, the phone would vibrate slightly in response to the user's activation of an on-screen control, making up for the lack of a normal tactile response that the user would experience when pressing a physical button. The resistive force that some “force feedback” joysticks and video game steering wheels provide is another form of haptic feedback.

This would allow a wireless glove to also be used as an output device, and would support finger bend analysis concurrently with hand motion analysis. Its accelerometers may be used to estimate the relative position of a user's finger tips. This finger tip position data may then be utilized by inverse kinematics algorithms to estimate the position of each finger joint and recreate the movements of the fingers of the user. The linear XYZ-translations vector generated by the hand of the user may be captured together with finger pressure and allows for recreating in real-time all of the gesture and posture performed by the user.

A shirt-based form factor of sensing system 100 (or “wireless shirt”) may incorporate wireless “edge-based processing” MEMS based sensors that are unobtrusively embedded in the fabric of an undershirt garment to facilitate monitoring and capturing physiological signal data from patients in the field over extended periods of time. This wearable technology will support patients undergoing for example, disease management, rehabilitation, etc. The wireless shirt allows clinicians to gather data from the home and community settings.

Current systems with multiple sensors for physical rehabilitation feature unwieldy wires between electrodes and the monitoring system. These wires limit the patient's activity and level of comfort and thus negatively influence the measured results. There are wireless telemetric devices that use wireless communication channels exclusively to transfer raw data from sensors to the monitoring station, or use standard high-level wireless protocols such as Bluetooth that are too complex, power demanding, and prone to interference by other devices operating in the same frequency range. Such characteristics limit their use for prolonged wearable monitoring.

The wireless shirt “edge-based processing” increases system processing power which allows for sophisticated real-time data processing within the confines of this wearable system. As a result, this wearable system supports real-time biofeedback, warnings, and event processing. Biofeedback techniques are important for physical medicine and rehabilitation. Intensive rehabilitation practice schedules are known to be important for recovery of motor function. Optimal approaches to rehabilitation involving extensive therapist-supervised motor training where individuals are typically seen as outpatients.

The wireless shirt and biofeedback systems are an alternative, as they reduce the extensive time to set-up a patient before each session and require limited time involvement of physicians and therapists. The wireless shirt can potentially address the time required to set up a patient for the required procedures because currently, tethered sensors need to be positioned on the patient, attached to the equipment, and a computer application needs to be started before each session.

The wireless shirt can keep the sensors positioned on the patient for prolonged periods, eliminating the need to position them for every training session. A computer or even a powerful smartphone/PDA can initiate a new training session whenever the patient is ready. The wireless shirt will support home rehabilitation and clinical settings while providing timely warnings the patient and specialized alerts to medical response services in the event of significant deviations of the norm or medical emergencies.

The wireless shirt “edge-based processing” utilizes MEMS based sensors, 32-bit processors with integrated wireless IEEE 802.15.4 compliant transceivers that run application-specific signal conditioning and algorithmic processing routines. One specific wireless shirt application may be used for sleep apnea monitoring and assessment. In this application, a number of bio-amplifiers and a three-dimensional accelerometer may be integrated on a flex-PCB design that allows for flexibility and comfort. Six flex-PCBs (“wireless processor pods”) may then be embedded into the undershirt garment material. Two wireless processor pods may be located near each pectoral area, two more may be located on the left and right side of the abdomen, and two more may be located by each shoulder blade. The wireless shirt may, thus, monitor position/orientation and activity of upper and lower extremities, specifically the thorax/abdomen regions. This will allow for monitoring respiration frequency and cessation events, besides assessing metabolic rate and cumulative energy expenditure.

The wireless shirt “edge-based processing” will support physiological sensors that includes ECG (electrocardiogram) sensors for monitoring heart activity, EMG (electromyography) sensor for monitoring muscle activity, a blood pressure sensor, etc. The multiple physiological sensor signals may be processed at wireless processor pod locations in a “sensor fused” fashion, so as to provide very efficient critical data and event delivery to collection/monitoring facilities. The wireless processor pods continuously collect and process bio-signal data and send critical/event data to the collection/monitoring facility. The wireless processor pods may also run various algorithms that perform bio-signal data acquisition, digital signal processing, motion/pressure analysis, and use multi-layer neural networks, as an example to model a patient's condition/state and activity.

A bracelet-based form factor of sensing system 100 is also possible. The bracelet device allows for wireless autonomous user mobility detection, monitoring and analysis. Specifically, the bracelet device allows for detecting, monitoring and profiling/correlating user mobility such as signaling through gestures and following hand motion commands. These captured movements may then be used for example, as input for console/PC games as directed user feedback.

The bracelet device can detect specific mobility events when required for purposes of critical event processing such as out-of-band signaling. The bracelet device sends event messages to a collector facility. The bracelet device requires no interaction from the user since the system is autonomous in its event processing.

The bracelet device can also monitor actual distance covered by the bracelet device user. Movement of any distance within any or all of the three dimensions pre-determined can be tracked over any specified time period. The bracelet device in conjunction with the collection node, is able to profile and correlate the spatial-temporal dynamics of the user wearing the bracelet device. This real-time/heuristic motion data will allow for the measuring and detection of motion related events correlated with, for example, specific console/PC game play.

The bracelet device supports capturing heart rate via the bracelet's wrist strap. A bracelet device wrist strap may also incorporate the XPU pressure transducer material. By utilizing very high input impedance BiFET operational amplifiers and the bracelet's processor core, the user's heart rate may be captured for console/PC game hepatic feedback via the bracelet's wireless transceiver.

The bracelet device contains a micro-controller processor unit (MPU), a micro electro mechanical system (MEMS) based three-dimensional accelerometer, using a wireless sensor network transceiver to communicate three dimensional accelerometer motion data to the collection node attached to a securely attached internet-enabled PC. Motion analysis software running on the collection node determines normal motion versus abnormal situations such as falls, violent shaking and/or tremors.

The bracelet device also contains a three dimensional accelerometer, where each dimension X, Y and Z is used to measure motion. The system implements a differential acceleration time-derivative algorithm with heuristic functionality. The output of the acceleration axes may be sampled with a 10-bit Analog Digital Converter (ADC). The wireless bracelet device measures five acceleration vectors per second for the three dimensions of possible movement. These acceleration vectors are sent via the wireless IEEE 802.15.4 link to the collector node. The acceleration vectors are signal averaged using weighted and/or not-weighted dynamically sized moving average convolution filters and used to determine distances traversed.

Analytics are available on the collection node that can be executed when required to determine motion “groups” (gestures, rollovers, spins, etc.) and this can be used as input to calculate the differential acceleration time derivatives to determine three dimensional shake and gesture events. The collection node performs three dimensional double integrations five times a second where the Path (x,y,z,t)≈={Sum(Ax·[t**2/2])+Sum(Ay·[t**2/2])+Sum(Az·[t**2/2])+Cx+Cy+Cz} and the integration results are summed and accumulated over the entire observation and monitoring period to provide location data as it relates to the bracelet device and the user.

For extreme data reliability, the bracelet device/collection node may incorporate the wireless IEEE 802.15.4 ZigBee mesh network technology standard for the best protection against failure. By placing the wireless IEEE 802.15.4 ZigBee receivers and transmitters in groups, the mesh network that results provides redundant paths to ensure alternate data path routes exist and there is no signal point of failure should a node fail. Wireless IEEE 802.15.4 ZigBee routers (extra specialized software running in the node) are used to greatly extend the range of the network by acting as relays for nodes that are too far apart to communicate directly.

The software architecture for the bracelet device's SoC (System on a Chip) uses an interrupt-driven architecture. The interrupt routines include the reading of the ADC (Analog Digital Converter), timers for creating the sampling frequency and handling interrupts from the IEEE 802.15.4 RF Transceiver. There a number of interrupt handlers that process data asynchronously from the non-interrupt main loop routine described before. The first is the Timer interrupt routine which is used as a time base and generates the sampling rate frequency used by the ADC. The second is the ADC interrupt routine which occurs when the ADC conversion of the three acceleration vectors Ax, Ay, Az is complete. It formats the ADC readings for read by the non-interrupt main processing loop. The third is the wireless bracelet device's RF transceiver status and data transfers interrupt handler. RF transceiver status/data transfers interrupt handler is used to process wireless bracelet device's RF transceiver events, transmit acceleration (Ax, Ay, Az) data/link energy data via wireless bracelet device's RF transceiver to the collection node and receive control/acknowledgment data via the bracelet device's RF transceiver from the collection node.

While various embodiments of the present invention have been described above, it should be understood that they have been presented by way of example only, and not limitation. Thus, the breadth and scope of the present invention should not be limited by any of the above-described exemplary embodiments, but should instead be defined only in accordance with the following claims and their equivalents.

What is claimed is:

1. A sensing system, comprising:
a transducer to measure pressure at each of a plurality of points in an area of interest, said transducer comprising:
a compressible layer, and
first and second flexible conductive layers, between which the compressible layer is disposed; and
means for transmitting data corresponding to said measured pressures.

2. The system according to claim 1, wherein said transmitting means comprises a transceiver.

3. The system according to claim 2, wherein said transceiver comprises a wireless transceiver.

4. The system according to claim 1, wherein each of said first and second flexible conductive layers comprises an electrode grid.

5. The system according to claim 4, further comprising a selector to turn on and off selected points of said electrode grid to variably measure pressure from said selected points within said area of interest.

6. The system according to claim 5, wherein said selector turns on and off said selected points of said electrode grid dynamically in real-time.

7. The system according to claim 1, wherein said plurality of points of interest comprise a plurality of parts of a foot selected from the group consisting of a forefoot area, a midfoot area, and a hindfoot area.

8. The system according to claim 7, wherein said group further comprises one or more of a plurality of phalanges, one or more of a plurality of metatarsals, one or more of a plurality of phalangeal joints, a ball of said foot, one or more of a plurality of tarsal bones forming an arch of said foot, a plantar fascia, a talus, calcaneus, and a subtalar joint.

9. The system according to claim 1, further comprising a data compressor to compress said data corresponding to said measured pressures before transmission by said transmitting means.

10. The system according to claim 1, wherein said transducer is embedded in a shoe sole.

11. The system according to claim 1, wherein said compressible material comprises a compressible conductive foam.

12. The system according to claim 9, wherein said compressible conductive foam comprises a material suitable for electrostatic discharge (ESD).

13. The system according to claim 1, further comprising a computer adapted to wirelessly receive said transmitted data and output the received data in a user-readable format.

14. The system according to claim 13, further comprising:
an accelerometer adapted to measure acceleration of said plurality of points within said area of interest; and
means for transmitting data corresponding to said measured accelerations.

15. The system according to claim 14, wherein said accelerometer is adapted to measure acceleration of each of said plurality of points within said area of interest along an x-axis, a y-axis, and a z-axis.

16. The system according to claim 15, wherein said computer further comprises means for integrating said transmitted data corresponding to acceleration of each of said plurality of points within said area of interest along said x-axis, said y-axis, and said z-axis.

17. The system according to claim 16, wherein said integrating means is adapted to output data corresponding to a velocity of each of said plurality of points within said area of interest along said x-axis, said y-axis, and said z-axis.

18. The system according to claim 16, wherein said integrating means is adapted to output data corresponding to a displacement of each of said plurality of points within said area of interest along said x-axis, said y-axis, and said z-axis.

19. The system according to claim 16, wherein said computer further comprises:
means for collecting said integrated data; and
means for correlating said collected data with said transmitted data corresponding to said measured pressures.

20. The system according to claim 19, further comprising an electronic game coupled to correlating means and adapted to receive said correlated data and interactively adapt said electronic game accordingly.

21. The system according to claim 19, wherein said computer further comprises diagnostic means for interpreting said collected and correlated data and thereby recommends changes to the positions of said plurality of points.

22. The system according to claim 21, wherein said computer further comprises means for designing an orthotic to make said recommended changes.

23. The system according to claim 16, wherein said computer further comprises tracking means for interpreting said collected and correlated data and thereby recommends changes to a training program.

24. The system according to claim 16, wherein said computer further comprises tracking means for interpreting said collected and correlated data and thereby recommends changes to a therapeutic program.

25. The system according to claim 13, wherein said user-readable format comprises an extensible markup language (XML).

26. A method for sensing a force applied to a first moving object by a second moving object in contact with the first object, comprising:
measuring the force at each of a plurality of points in an area of interest between the first and second objects;
activating, dynamically in real-time, selected points within said area of interest to variably measure force at said selected points;
measuring an acceleration of each of said plurality of points within said area of interest along an x-axis, a y-axis, and a z-axis;
transmitting data corresponding to said measured forces and said measured accelerations to a computer adapted to receive said transmitted data and output the received data in a user-readable format;
integrating said transmitted data corresponding to acceleration of each of said plurality of points within said area of interest along said x-axis, said y-axis, and said z-axis;
collecting said integrated data; and
correlating said collected data with said transmitted data corresponding to said measured forces and said measured accelerations.

27. The method according to claim 26, further comprising outputting data corresponding to a velocity of each of said plurality of points within said area of interest along said x-axis, said y-axis, and said z-axis.

28. The method according to claim 26, further comprising outputting data corresponding to a displacement of each of said plurality of points within said area of interest along said x-axis, said y-axis, and said z-axis.

29. The method according to claim 26, further comprising compressing said data corresponding to said measured forces before said transmitting step.

30. The method according to claim 26, further comprising:
coupling an electronic game coupled to receive said correlated data; and
interactively adapting said electronic game in accordance with said correlated data.

31. The method according to claim 26, further comprising:
diagnostically interpreting said collected and correlated data; and
changing the positions of said plurality of points in accordance with said interpretations.

32. The method according to claim 26, further comprising:
establishing a training program of predetermined movements of said plurality of points;
diagnostically interpreting said collected and correlated data;
tracking said interpretations of said collected and correlated data as a function of time; and
changing said predetermined movements of said plurality of points in accordance with said tracked interpretations.

33. The method according to claim 26, further comprising:
establishing a therapeutic program of predetermined movements of said plurality of points;
diagnostically interpreting said collected and correlated data;
tracking said interpretations of said collected and correlated data as a function of time; and
changing said predetermined movements of said plurality of points in accordance with said tracked interpretations.

34. A computer-readable medium comprising computer-executable instructions, the medium comprising:
one or more instructions for measuring the force at each of a plurality of points in an area of interest between the first and second objects;
one or more instructions for activating, dynamically in real-time, selected points within said area of interest to variably measure force at said selected points;
one or more instructions for measuring an acceleration of each of said plurality of points within said area of interest along an x-axis, a y-axis, and a z-axis;
one or more instructions for transmitting data corresponding to said measured forces and said measured accelerations to a computer adapted to receive said transmitted data and output the received data in a user-readable format;
one or more instructions for integrating said transmitted data corresponding to acceleration of each of said plurality of points within said area of interest along said x-axis, said y-axis, and said z-axis;
one or more instructions for collecting said integrated data; and
one or more instructions for correlating said collected data with said transmitted data corresponding to said measured forces and said measured accelerations.

35. The medium according to claim 34, further comprising one or more instructions for outputting data corresponding to a velocity of each of said plurality of points within said area of interest along said x-axis, said y-axis, and said z-axis.

36. The medium according to claim 34, further comprising one or more instructions for outputting data corresponding to a displacement of each of said plurality of points within said area of interest along said x-axis, said y-axis, and said z-axis.

37. The medium according to claim 34, further comprising one or more instructions for compressing said data corresponding to said measured forces before said transmitting step.

38. The medium according to claim 34, further comprising:
one or more instructions for coupling an electronic game coupled to receive said correlated data; and
one or more instructions for interactively adapting said electronic game in accordance with said correlated data.

39. The medium according to claim 34, further comprising:
one or more instructions for diagnostically interpreting said collected and correlated data; and
one or more instructions for changing the positions of said plurality of points in accordance with said interpretations.

40. The medium according to claim 34, further comprising:
one or more instructions for establishing a training program of predetermined movements of said plurality of points;
one or more instructions for diagnostically interpreting said collected and correlated data;
one or more instructions for tracking said interpretations of said collected and correlated data as a function of time; and
one or more instructions for changing said predetermined movements of said plurality of points in accordance with said tracked interpretations.

41. The medium according to claim 34, further comprising:
one or more instructions for establishing a therapeutic program of predetermined movements of said plurality of points;
one or more instructions for diagnostically interpreting said collected and correlated data;
one or more instructions for tracking said interpretations of said collected and correlated data as a function of time; and
one or more instructions for changing said predetermined movements of said plurality of points in accordance with said tracked interpretations.
